(19) United States
(12) Patent Application Publication
(10) Pub. No.: US 2023/0241141 A1
(43) Pub. Date: Aug. 3, 2023

(54) PHARMACEUTICAL COMPOSITION FOR PREVENTING OR TREATING CANCER

(71) Applicant: VIROCURE, INC., Seoul (KR)

(72) Inventors: Haeng Jun YOO, Seoul (KR); Sang Kyoung HAN, Seoul (KR); Yeon Sook LEE, Gunpo-si, Gyeonggi-do (KR); Chan KIM, Yongin-si, Gyeonggi-do (KR); Hong Jae JEON, Seongnam-si, Gyeonggi-do (KR)

(73) Assignee: VIROCURE, INC., Seoul (KR)

(21) Appl. No.: 17/999,371

(22) PCT Filed: Apr. 7, 2021
(86) PCT No.: PCT/KR2021/004323

§ 371 Date: Nov. 18, 2022

Related U.S. Application Data
(60) Provisional application No. 63/027,574, filed on May 20, 2020.

(51) Int. Cl. A61K 35/765; A61K 39/395; A61P 35/00; A61P 1/00; A61P 17/00; A61P 13/12

(52) U.S. Cl. CPC A61K 35/765; A61K 39/3955; A61P 35/00; A61P 1/00; A61P 17/00; A61P 13/12

(57) ABSTRACT

Provided are a pharmaceutical composition including reovirus or reovirus-treated biological sample as an active ingredient for prevention or treatment of cancer, and a method for prevention or treatment of cancer using same. In addition, provided are a combination use of the pharmaceutical composition and an immune checkpoint inhibitor for treatment of cancer.

### TECHNICAL FIELD

The present invention relates to a pharmaceutical composition for preventing or treating cancer, and a method of preventing or treating cancer using the same. In addition, the present invention relates to a use of the pharmaceutical composition and an immune checkpoint inhibitor in a combined treatment for cancer.

This application claims priority to and the benefit of U.S. Provisional Application No. 63/027,574, filed on May 20, 2020, the disclosure of which is incorporated herein by reference in its entirety.

### BACKGROUND ART

Cancer is generally defined as diseases, malignant tumors and neoplasms in which cells divide at an excessive rate and their functions become abnormal. Standard treatments include surgery, chemotherapy and radiation therapy to remove affected cancerous tissue. Certain cancers do not respond or become resistant to such chemotherapy, radiation therapy, and other treatments. While some human tumors are sensitive to conventional chemo/radiation therapy, various solid tumors (e.g., colorectal, brain, breast, ovarian and other tumors) and hematological tumors are known to be refractory to conventional treatment regimens. Recently, unlike the conventional cancer treatments, immunotherapy, which uses the immune system to treat cancer, is drawing attention.

Immunochemotherapy is a method that induces immune cells to selectively attack only cancer cells by stimulating the immune system by injecting an artificial immune protein into the body, unlike conventional anticancer agents, and may be largely classified into passive immunotherapy and active immunotherapy. Passive immunotherapy includes an immune checkpoint inhibitor, an immune cell therapy, and a therapeutic antibody. Among these, immune checkpoint inhibitors are drugs that block the activation of an immune checkpoint protein involved in T cell suppression to activate T cells and attack cancer cells, and include CTLA-4, PD-1, and PD-L1 inhibitors. In 2016, a PD-L1 antibody drug (atezolizumab) was approved by the FDA for anticancer treatment, but there is a limitation in that it shows limited therapeutic effect as a single treatment of an immune checkpoint inhibitor. In addition, active immunotherapy includes therapeutic cancer vaccines and immune-modulating agents, and among these, therapeutic cancer vaccines are prepared from cancer cells or cancer cell-derived substances, and are drugs that are injected into the human body to make the natural defense system of the body operable. However, there are problems in that therapeutic cancer vaccines have complicated production processes, are difficult to apply to various types of cancer, and impose a financial burden on patients since they are a personalized therapy.

Therefore, it is important to develop a more effective approach, particularly, a combination approach, for improving the prevention and treatment of cancer.

Colorectal cancer is a malignant tumor composed of cancer cells in the colon, and has symptoms such as a change in bowel habit, diarrhea, constipation, bloody stool, abdominal pain, abdominal distension, fatigue, the loss of appetite, and indigestion. For colorectal cancer treatment, the treatment method is determined according to the degree of tissue penetration of cancer cells, and in most cases of primary cancer, resection is combined with a chemotherapy or radiation therapy. However, as side effects of surgery, pulmonary complications caused by general anesthesia after surgery, anastomotic leak, bleeding, or intestinal obstruction may occur. In addition, the side effects of chemotherapy include leukopenia or thrombopenia, hair loss, nausea (sickness), vomiting, and fatigue, and side effects of radiation therapy include pelvic pain, a change in bowel habit, dysuria, anal pain, diarrhea, or hair loss.

In addition, colorectal cancer recurs approximately 20 to 50% even after radical resection, and appears in three types, such as local recurrence, distant metastasis, and local recurrence with distant metastasis. In general, there are wide-range of recurrences accompanied by local recurrence and distant metastasis, and when metastasized, stage 4 colorectal cancer, classified as the most advanced state, is known to have poor prognosis. Accordingly, there is a need for research on a novel treatment method for compensating for a current therapeutic method with many side effects and effectively treating colorectal cancer.

Meanwhile, viruses are one of the biotherapeutic agents and have the concept of targeted therapeutics that attack tumor cells using genetic mutations therein. Anticancer viruses selectively proliferate in cancer cells, and induce tumor necrosis and death.

Oncolytic viruses are viruses used in cancer treatment, and an anticancer therapy using these viruses is called oncolytic viral (OV) therapy. Research on cancer treatments using wild-type oncolytic viruses is distinguished from gene therapy, which is to mainly see the oncolytic effect caused by the expression of a therapeutic gene by inserting an existing therapeutic gene into a virus, started when it became known that some wild-type viruses have an intrinsically potent oncolytic ability.

It has been long time reported that cancer is naturally cured due to natural infection by various types of viruses, and since the research on the tumor-specific lytic mechanism by wild-type viruses began in earnest, cancer treatment research using a wild-type reovirus has reached a phase 3 clinical trial. In addition, adenoviruses, polioviruses, herpes simplex viruses, and vesicular stomatitis viruses have been developed, and methods to increase the efficacy and stability of the viruses are under investigation.

### DISCLOSURE

### Technical Problem

The inventors confirmed that not only cancer can be treated through oral administration of reoviruses, but the combined use of the viruses with an immune checkpoint inhibitor shows a significant synergistic effect on cancer treatment, thereby improving the anticancer effect. Thus, the present invention was completed.

Therefore, the present invention is directed to providing a pharmaceutical composition for preventing or treating cancer, which includes a reovirus or a reovirus-treated biological sample as an active ingredient.

The present invention is also directed to providing a pharmaceutical composition for preventing or treating recurrent cancer, which includes a reovirus or a reovirus-treated biological sample.

However, technical problems to be solved in the present invention are not limited to the above-described problems, and other problems which are not described herein will be fully understood by those of ordinary skill in the art from the following descriptions.

### Technical Solution

To achieve the purposes of the present invention, the present invention provides a pharmaceutical composition for preventing or treating cancer, which includes a reovirus or a reovirus-treated biological sample as an active ingredient. In addition, the present invention provides a pharmaceutical composition for preventing or treating recurrent cancer, which includes a reovirus or a reovirus-treated biological sample as an active ingredient.

In addition, the present invention provides a method of preventing or treating cancer or recurrent cancer, which includes administering a composition including a reovirus or a reovirus-treated biological sample as an active ingredient into a subject.

Moreover, the present invention provides a use of a composition including a reovirus or a reovirus-treated biological sample as an active ingredient for prevention or treatment of cancer or recurrent cancer.

Moreover, the present invention provides a use of a composition including a reovirus or a reovirus-treated biological sample as an active ingredient for preparation of a drug for preventing or treating cancer or recurrent cancer.

In one embodiment of the present invention, the cancer may be one or more selected from the group consisting of cervical cancer, lung cancer, pancreatic cancer, non-small cell lung cancer, liver cancer, colon cancer, colorectal cancer, bone cancer, skin cancer, head and neck cancer, skin melanoma, intraocular melanoma, uterine cancer, ovarian cancer, rectal cancer, brain cancer, blood cancer, stomach cancer, perianal cancer, breast cancer, fallopian tube carcinoma, endometrial carcinoma, vaginal cancer, vulvar carcinoma, Hodgkin's disease, esophageal cancer, small intestine cancer, endocrine gland cancer, thyroid cancer, parathyroid cancer, adrenal cancer, soft tissue sarcoma, urethral cancer, penile cancer, prostate cancer, bladder cancer, kidney cancer, urinary tract cancer, renal cell carcinoma, renal pelvic carcinoma, CNS central nervous system (CNS) tumors, primary CNS lymphoma, spinal cord tumors, brainstem glioma, and pituitary adenoma, but the present invention is not limited thereto.

In another embodiment of the present invention, the pharmaceutical composition may further include an immune checkpoint inhibitor, but the present invention is not limited thereto.

In still another embodiment of the present invention, the immune checkpoint inhibitor may be one or more selected from the group consisting of a PD-L1 inhibitor, a PD-1 inhibitor, and a CTLA-4 inhibitor, but the present invention is not limited thereto.

In yet another embodiment of the present invention, the reovirus or reovirus-treated biological sample, and the immune checkpoint inhibitor may be simultaneously, separately or sequentially administered, but the present invention is not limited thereto.

In yet another embodiment of the present invention, the reovirus or reovirus-treated biological sample may be orally administered, but the present invention is not limited thereto.

In yet another embodiment of the present invention, the pharmaceutical composition may increase the infiltration of CD8+ T cells into a tumor, but the present invention is not limited thereto.

In yet another embodiment of the present invention, the immune checkpoint inhibitor may be parenterally administered, but the present invention is not limited thereto.

In yet another embodiment of the present invention, the pharmaceutical composition may be administered simultaneously, separately or sequentially with the immune checkpoint inhibitor, but the present invention is not limited thereto.

In yet another embodiment of the present invention, the biological sample may be prepared to kill cancer cells by applying an effective amount of reovirus to an ex vivo biological sample, but the present invention is not limited thereto.

In yet another embodiment of the present invention, the biological sample may be a bone marrow sample, a fat-derived stem cell sample, or a blood sample, but the present invention is not limited thereto.

### Advantageous Effects

The present inventors not only confirmed the cancer treatment effect of reoviruses alone, but also confirmed significant increases in cancer treatment effects such as tumor volume reduction, inhibition of tumor growth rate, and an increase in survival rate for colorectal cancer, skin cancer, and renal cancer when reoviruses are orally administered and used in combination with an immune checkpoint inhibitor, and particularly, when a PD-1 antibody, a CTLA-4 antibody, and reoviruses are used in combination, the prevent inventors confirmed that not only complete regression of cancer, but also the recurrence of cancer can be inhibited. Therefore, it is expected that the reovirus of the present invention will be usefully used as a therapeutic agent and therapeutic adjuvants for various types of cancer.

### DESCRIPTION OF DRAWINGS

FIG. 1 shows the process of producing azoxymethane (AOM)/dextran sulfate sodium (DSS)-induced colorectal cancer animal models and the administration schedule of a reovirus (RC402) and an anti-PD-L1 antibody.

FIG. 2 shows disease activity index (DAI) determination criteria for measuring the severity of a colorectal cancer-related disease of a colorectal cancer animal model.

FIG. 3 shows the DAI measurement results of a normal control, an AOM/DSS-induced colorectal group (vehicle), a reovirus oral administration group (RC402), an immune checkpoint inhibitor abdominal administration group (αPD-L1), and a reovirus/immune checkpoint inhibitor coadministration group (RC402+αPD-L1).

FIG. 4 shows the survival rate measurement results of a normal control, an AOM/DSS-induced colorectal group (vehicle), a reovirus oral administration group (RC402), an immune checkpoint inhibitor abdominal administration group (αPD-L1), and a reovirus/immune checkpoint inhibitor coadministration group (RC402+αPD-L1).

FIG. 5 shows the administration schedule of a reovirus (RC402) and an anti-PD-L1 antibody in a colorectal cancer animal model using CT26 colorectal cancer cell implantation.

FIG. 6 shows the survival rate measurement result according to the administration schedule of FIG. 5.

FIG. 7 shows the administration schedule of a reovirus (RC402) and an anti-PD-L1 antibody in skin cancer animal model using B16F10 melanoma cell implantation.

FIG. 8 shows the result of measuring a tumor volume according to the administration schedule of FIG. 7.

FIG. 9 shows the survival rate measurement result according to the administration schedule of FIG. 7.

FIG. 10 shows the reovirus-only administration schedule in renal cancer animal model using RENCA renal cancer cell implantation.

FIG. 11 shows the results of measuring a tumor volume and a growth rate according to the administration schedule of FIG. 10.

FIG. 12 shows the reovirus-only administration schedule in colorectal cancer animal model using MC38 colon adenocarcinoma cell implantation.

FIG. 13 shows the result of measuring a tumor volume and the survival rate measurement result according to the administration schedule of FIG. 12.

FIG. 14 shows the proportion of CD8+ T cells infiltrated into a tumor according to reovirus (RC402) administration in an MC38-implanted colorectal cancer animal model.

FIG. 15 shows the results of observing the degree of CD8+ T cell infiltration according to reovirus (RC402) administration into a tumor and a lymph node obtained from an MC38-implanted colorectal cancer animal model by a fluorescent microscope after immunohistochemical staining and DAPI staining.

FIG. 16 shows the result of obtaining the infiltration of CD8+ T cells into a tumor according to reovirus (RC402) administration in the tumor obtained from an MC38-implanted colorectal cancer animal model by a fluorescent microscope after immunohistochemical staining and DAPI staining.

FIG. 17 shows the administration schedules of a reovirus (RC402), and an anti-PD-1 antibody and/or an anti-CTLA-4 antibody in a colorectal cancer animal model using CT26 colorectal cancer cell implantation.

FIG. 18 shows the result of measuring a tumor volume and the survival rate measurement result according to the administration schedule of FIG. 17.

FIG. 19 shows the result of observing the degree of CD8+ T cell infiltration according to the administration of a reovirus (RC402), and an anti-PD-1 antibody and/or an anti-CTLA-4 antibody in a tumor obtained from a CT26-implanted colorectal cancer animal model through immunohistochemical staining and flow cytometry.

FIG. 20 shows the result of the re-implantation of CT26 after complete regression of a tumor in order to confirm the effect of preventing the recurrence of CT26 colorectal cancer.

FIG. 21 shows the flow cytometry result showing the proportion of memory T cells (CD44+CD62+CD8+) in the spleen taken from a CT26 re-implanted mouse after complete regression.

FIG. 22 shows the flow cytometry result showing the proportion of CD8+ T cells expressing a T cell receptor using an MC38-specific antigen peptide-loaded tetrameric synthetic MHC complex.

FIG. 23 shows the PD-1 expression of CD8+ cells according to reovirus (RC402) administration in a mesenteric lymph node and blood in an MC38 colorectal cancer model.

### MODES OF THE INVENTION

The present invention provides a pharmaceutical composition for preventing or treating cancer or recurrent cancer, which includes a reovirus or a reovirus-treated biological sample as an active ingredient.

Hereinafter, the present invention will be described in detail.

The term “reovirus” used herein refers to a double-stranded virus with a segmented RNA genome, and any virus classified in the family of Reoviridae. The virion of a reovirus has a diameter of 60 to 80 nm, and has two concentric capsid shells. This genome consists of double-stranded RNA with 10 to 12 discontinuous segments, and has a total genome size of 16 to 27 kbp, and each RNA segment has a different size.

In the present invention, the reovirus includes not only a naturally occurring reovirus, but also a modified or recombinant reovirus. When a reovirus can be isolated from the nature and has not been artificially modified by a human, the reovirus is “naturally occurring.” For example, the reovirus may be derived from a “field source,” that is, a human infected by the reovirus.

The reovirus may be modified, but may lytically infect mammalian cells having an activated ras pathway. In addition, the reovirus may be chemically or biochemically (e.g., treated using a protease such as chymotrypsin or trypsin) pretreated before administration into proliferating cells. The infectivity of the virus may be improved by removing the envelope or capsid of the virus through pretreatment using a protease. The reovirus may be coated with liposomes or micelles, and for example, to produce new infectious subviral particles, a virion may be treated by chymotrypsin in the presence of an alkyl sulfate detergent at a micelle-forming concentration.

In the present invention, the reovirus may be a wild-type reovirus or an attenuated reovirus, but the present invention is not limited thereto.

The attenuated reovirus includes an infectious, replicable reovirus virion lacking reovirus sigma-1 capsid protein that can be detected by the genome of a reovirus lacking the S1 gene of a wild-type reovirus. As such, attenuated reoviruses stem from the surprising observation in that a mutated reovirus lacking detectable reovirus sigma-1 capsid protein desirably avoids a cytopathic effect on non-malignant cells while unexpectedly maintaining the ability to productively infect target tumor cells. As mentioned above, prior to the present disclosure, it was understood that particles of a sigma-1-deficient reovirus are non-infectious.

In a specific embodiment, the attenuated reovirus may include the S4 gene of a mutated reovirus. The reovirus wild-type S4 gene encodes the reovirus capsid sigma-3 polypeptide involved in virion processing during the reoviral transfection of host cells.

In a specific embodiment, the attenuated reovirus may include a mutated reovirus S4 gene including one or more mutations in the genome sequence encoding the reovirus sigma-3 polypeptide, compared with the sequence of the wild-type S4 gene.

The attenuated reovirus lacks a detectable sigma-1 capsid protein, but is unexpectedly infectious. As described above, it is shown that sigma-1 is associated with reovirus binding and attachment to cells via a cell surface sialic acid residue in the early stage of virus transfection. Despite lacking detectable sigma-1, the attenuated reovirus disclosed herein is capable of host cell entry and the replication of cytolytic viruses. In addition, the attenuated reovirus exhibits a remarkable characteristic of inducing a declined level (that is, decreased statistical significance) of one or more cytopathic effects on non-malignant cells, compared with the level of a cytopathic effect on non-malignant cells, which is caused by a naturally-occurring non-attenuated reovirus.

An attenuated reovirus may be derived from any reovirus, meaning a member of Reoviridae, and include reoviruses with various affinities, which may be obtained from various sources.

In a specific embodiment, the attenuated reovirus may be a mammalian reovirus, and in another embodiment, a human reovirus. In another embodiment (e.g., to be used in an animal model having relevance with a human disease or to be used in veterinary-related applications), the attenuated reovirus may be derived from one or more reoviruses having affinity to cells of different mammalian species, including non-human primates (e.g., chimpanzees, gorillas, macaques, monkeys, etc.), rodents (e.g., mice, rats, gerbils, hamsters, rabbits, guinea pigs, etc.), dogs, cats, common livestock (e.g., cattle, horses, pigs, and goats), or alternatively, a reovirus with distinct affinity (e.g., avian reovirus) may be used.

The attenuated reovirus involves the generation and identification of sigma-1-deficient and/or sigma-1-defective mutant(s) by molecular biology approaches (in a specific embodiment, additionally or alternatively, including the generation and identification of sigma-3-deficient and/or sigma-3-defective mutant(s)), and in addition, may be derived according to other methologies, including artificial induction of such sigma-1 (and/or sigma-3) mutant(s) by the isolation of naturally-occurring sigma-1-deficient and/or sigma-1-defective mutant(s) and/or sigma-3 mutant(s), and/or chemical, physical and/or genetic techniques (e.g., selective recombination of a reovirus gene in productively infected host cells).

The attenuated reovirus includes infectious, replicable reovirus virions lacking the wild-type reovirus S1 gene and consequently lacking a detectable reovirus sigma-1 capsid protein (that is, viral particles including the genome, core protein and protein envelope of a virus).

In a specific embodiment, the attenuated reovirus lacks the wild-type reovirus S4 gene and expresses mutated reovirus sigma-3 capsid protein. As known in the related art, an infectious, replicable reovirus is one that can bind to host cells and be internalized therein for a sufficient time under proper conditions, and in addition, indicates the replication of the reoviral genome and biosynthesis of the structural protein of reovirus in the manner of allowing the assembly of a complete progeny reovirus that can productively infect other host cells for perpetuating the replication cycle of the virus when released from the host cells.

For example, attenuated reoviruses for cancer treatment are disclosed in US Patent Application Publication Nos. US2009/0214479 and US2009/0104162, each of which is incorporated herein by reference in its entirety.

The human reovirus may consist of three serotypes as follows: type 1 (Lang strain or T1L), type 2 (Jones strain, T2J) and type 3 (Dearing strain, T3D; or Abney strain, T3A). The three serotypes may be easily identified based on a neutralization reaction and hemagglutinin inhibition assay.

In the present invention, the reovirus may be obtained using baby hamster kidney (BHK) cells (e.g., BHK-21 cells), but the present invention is not limited thereto. In the term “BHK-21 cell” used herein, BHK is the abbreviation of Baby Hamster Kidney, and BHK cells were originally isolated by polyoma transformation of hamster cells, and may be used as a substrate, with respect to a vaccine, for virus propagation and virus-mediated expression. In addition, BHK cells are useful as a host cell line for stable expression of various recombinant proteins. BHK Strain 21 (HK-21) was derived from baby hamster kidneys of five unsexed, 1-day-old hamsters on March, 1961 by IA Macpherson and MGP Stoker. The hamsters were used to produce BHK-21 cells, and generally known as the Syrian golden hamster (Mesocricetus auratus).

The “biological sample” in the term “reovirus-treated biological sample” used herein is any biological sample obtained from a subject, a cell line, a tissue culture or other cell sources, and refers to, for example, adult stem cells (adipose-derived stem cells or bone marrow stem cells), or cord blood stem cells. Methods for obtaining tissue biopsies and body fluids from mammals are well known in the art. For example, the adipose-derived stem cells may be administered into a cancer patient after pre-treatment with an oncolytic virus.

In the present invention, the biological sample may be prepared to kill a plurality of cancer cells by applying an effective amount of reovirus with respect to an ex vivo biological sample, but the present invention is not limited thereto.

In the present invention, the biological sample may be a bone marrow sample, an adipose-derived stem cell sample, or a blood sample, but the present invention is not limited thereto.

The pharmaceutical composition of the present invention may be orally administered, but the present invention is not limited thereto. Oral administration may minimize side effects caused by repetitive administration. Specifically, in the case of 2 to 3 consecutive intravenous administrations of the same amount of virus, it may be fatal due to side effects such as a cytokine storm. In contrast, in the case of repetitive oral administration of the same amount or more of virus, such side effects may not occur.

In addition, the pharmaceutical composition for oral administration of the present invention can be administered in a naked form without enteric coating for enteral administration.

Further, the pharmaceutical composition for oral administration of the present invention may maximize an anticancer effect due to excellent viral delivery efficiency into cancer. The efficiency of viral delivery into cancer is considered to be the most important factor in determining an anticancer effect. Cancer treatment through intravenous administration of an anticancer virus has been attempted, but it has been reported that this administration method removes most of the virus by the antiviral immune system in the blood and thus greatly reduces the delivery efficiency into cancer. On the other hand, in the case of oral administration, the antiviral effect caused by the immune system is relatively low, so it is expected that the virus delivery efficiency will be good.

In the present invention, the pharmaceutical composition for oral administration may be co-administrated with an immune checkpoint inhibitor. 80 to 90% metastatic colorectal cancer urgently requires combination therapeutics, which a microsatellite stable (MSS) type not responding to an immune checkpoint inhibitor, for increasing and maximizing its reactivity. Since a reovirus has an excellent priming effect of converting such a carcinoma into a reactive carcinoma, when the pharmaceutical composition of the present invention is used in combination with an immune checkpoint inhibitor, it can be a breakthrough in cancer treatment through synergy that maximizes a rate of viral delivery by oral administration, and dramatically increases the reactivity of the immune checkpoint inhibitor.

The term “immune checkpoint inhibitor” used herein refers to a material that entirely or partially inhibits, interferes with or regulates one or more immune checkpoint proteins, also called cancer immunotherapy. Immune checkpoint proteins regulate the activation or function of T cells. A number of immune checkpoint proteins, for example, PD-1, PD-L1 and CTLA-4 are known (Nature Reviews Cancer 12: 252-264, 2012). These proteins are involved in the co-stimulatory or inhibitory interaction of T cell responses. The immune checkpoint inhibitor may include an antibody, and may be derived from an antibody.

In the present invention, the immune checkpoint inhibitor may be a drug that specifically binds to programmed cell death protein 1 (PD-1), but the present invention is not limited thereto. In one exemplary embodiment, the drug specifically binding to PD-1 may be an anti PD-1 antibody, which may be selected from the group consisting of nivolumab, pembrolizumab, cemiplimab, spartalizumab, camrelizumab, sintilimab, tislelizumab, toripalimab, dostarlimab, INCMGA00012, AMP-224, and AMP-514, but the present invention is not limited thereto.

In the present invention, the immune checkpoint inhibitor may be a drug that specifically binds to programmed death-ligand 1 (PD-L1), but the present invention is not limited thereto. In one exemplary embodiment, the drug specifically binding to PD-L1 may be an anti-PD-L1 antibody, which may be selected from the group consisting of atezolizumab, avelumab, durvalumab, envafolimab, cosibelimab, AUNP12, CA-170 and BMS-986189, but the present invention is not limited thereto.

In the present invention, the immune checkpoint inhibitor may be a drug that specifically binds to cytotoxic T-lymphocyte-associated protein 4 (CTLA-4), but the present invention is not limited thereto. In an exemplary embodiment, the drug specifically binding to CTLA-4 may be an anti-CTLA-4 antibody, which may be ipilimumab or tremelimumab, but the present invention is not limited thereto.

The term “antibody” used herein is a material that specifically binds to an immune checkpoint protein such as PD-1, PD-L1 or CTLA4 and exhibits inhibitory activity against an immune checkpoint. The scope of the antibodies includes a complete form of antibody as well as an antigen-binding site of an antibody molecule. In addition, the antibodies include monoclonal antibodies, human antibodies, humanized antibodies, and chimeric antibodies, but the present invention is not limited thereto.

The term “cancer” used herein refers to a disease associated with the regulation of cell death, and generated by excessive proliferation of cells when the balance of normal apoptosis is broken. Such abnormal, excessively-proliferated cells may, in some cases, infiltrate into peripheral tissue and organs and thus form a mass, causing destruction or deformation of the normal structure of the body, this condition is generally called a tumor.

Generally, the tumor may be classified into a benign tumor and a malignant tumor. Malignant tumors grow very rapidly compared to benign tumors, and infiltrate surrounding tissue to induce metastasis, thus threatening life. Such malignant tumors are generally referred to as “cancer.”

In the present invention, the cancer may be one or more selected from the group consisting of cervical cancer, lung cancer, pancreatic cancer, non-small cell lung cancer, liver cancer, colon cancer, colorectal cancer, bone cancer, skin cancer, head and neck cancer, skin melanoma, intraocular melanoma, uterine cancer, ovarian cancer, rectal cancer, brain cancer, blood cancer, stomach cancer, perianal cancer, breast cancer, fallopian tube carcinoma, endometrial carcinoma, vaginal cancer, vulvar carcinoma, Hodgkin's disease, esophageal cancer, small intestine cancer, endocrine gland cancer, thyroid cancer, parathyroid cancer, adrenal cancer, soft tissue sarcoma, urethral cancer, penile cancer, prostate cancer, bladder cancer, kidney cancer, urinary tract cancer, renal cell carcinoma, renal pelvic carcinoma, CNS central nervous system (CNS) tumors, primary CNS lymphoma, spinal cord tumors, brainstem glioma, and pituitary adenoma, but the present invention is not limited thereto.

The term “synergy” used herein means that, as shown in the literature (Chou and Talalay, Adv. Enzyme. Regul., 22:27-55, 1984), the effect generated when each component is administered in combination is greater than the sum of the effects generated when the pharmaceutical composition of the present invention is administered alone as a single component.

The term “administered in combination” used herein refers that different components are administered in combination into a subject. The co-administration of different components means that, to obtain a desired therapeutic effect, each component may be administered in any order at the same time or sequentially at different times.

The pharmaceutical composition in the present invention may include a reovirus and a PD-L1 inhibitor as active ingredients, but the present invention is not limited thereto. Alternatively, in the present invention, a reovirus-containing pharmaceutical composition may be administered in combination with a PD-L1 inhibitor, but the present invention is not limited thereto.

In the present invention, the pharmaceutical composition may include a reovirus and a PD-1 inhibitor as active ingredients, but the present invention is not limited thereto. Alternatively, in the present invention, a reovirus-containing pharmaceutical composition may be administered in combination with a PD-1 inhibitor, but the present invention is not limited thereto.

In the present invention, the pharmaceutical composition may include a reovirus, a PD-1 inhibitor, and a CTLA-4 inhibitor as active ingredients, but the present invention is not limited thereto. Alternatively, in the present invention, a reovirus-containing pharmaceutical composition may be administered in combination with a PD-L1 inhibitor and a CTLA-4 inhibitor, but the present invention is not limited thereto.

In the present invention, the pharmaceutical composition for oral administration may be administered simultaneously, separately or sequentially with an immune checkpoint inhibitor.

The immune checkpoint inhibitor may be administered into a subject via various routes. All methods for administration may be expected, and the pharmaceutical composition of the present invention may be administered by, for example, oral administration, intranasal administration, intratumoral administration, bronchobronchial administration, arterial injection, intravenous injection, subcutaneous injection, intramuscular injection, or intraperitoneal injection. A daily dose may be administered once or divided into several times a day.

In the present invention, the reovirus may include a heterologous gene for cancer treatment, but the present invention is not limited thereto. The term “heterologous gene” is used to mean accepting any gene that is not found in the viral genome. A heterologous gene may be an allelic variant of a wild-type gene or a mutant gene. The heterologous gene for cancer treatment may be inserted into an essential region or non-essential region in the reovirus gene to increase anticancer activity.

The heterologous gene may be operably linked to a control sequence that allows the above-described heterologous gene to be expressed in cells under in vivo conditions. Accordingly, the virus of the present invention may be used to deliver heterologous gene/genes into cells under in vivo condition in which the heterologous gene(s) can be expressed. The gene conventionally encodes a protein capable of enhancing the oncolytic property of the virus. The gene itself may be a cytotoxin, or may encode a protein that can promote/improve an antitumor immune response.

The heterologous gene may include all genes that treats cancer by insertion into a replicable oncolytic virus or an existing non-replicable virus vector.

The term “therapeutic gene” used herein is intended to describe all of the wide-range of genes whose expression affects a preferable result, for example, an anticancer effect. The reovirus of the present invention may include one or more target sequences encoding a therapeutic gene. The therapeutic gene may have pharmacological or prophylactic activity when properly administered to a patient, particularly, a patient suffering from a condition of a disease or disorder, or a patient to be protected from such a disease or condition.

Such pharmacological or prophylactic activity means that it is expected to be associated with a beneficial effect on the course or symptom of the disease or condition. The target sequence may be homologous or heterologous to the target cells into which the sequence is introduced, and encodes all or a part of a polypeptide, particularly, a therapeutic or prophylactic polypeptide imparting a therapeutic or prophylactic property. A polypeptide is understood as any translational product of a polynucleotide regardless of a size and glycosylation, and includes a peptide and a protein. Therapeutic polypeptides include polypeptides capable of compensating for a deleted or deficient protein in an animal or human organism or those for limiting or removing harmful cells from the body due to a toxic effect. They may also be an immunity-conferring polypeptide serving as an endogenous antigen to induce either a humoral or cellular response or both thereof, and include, for example, a drug-sensitizing gene, a proapoptotic gene, a cytostatic gene, a cytotoxic gene, a tumor suppressor gene, an antigenic gene, an anti-angiogenic gene, and a cytokine gene, but the present invention is not limited thereto.

The active ingredient of the present invention is administered into a subject at a pharmaceutically effective amount.

The term “pharmaceutically effective amount” used herein refers to an amount sufficient for treating a disease at a reasonable benefit or risk ratio applicable for medical treatment, and an effective dosage may be determined by parameters including a type of a patient's disease, severity, drug activity, sensitivity to a drug, administration time, an administration route and an excretion rate, the duration of treatment and drugs simultaneously used, and other parameters well known in the medical field.

An effective amount of the virus is a dose required for a sufficient time to alleviate, improve, mitigate ameliorate, stabilize a disease, inhibit the spread of the disease, delay the progression of a disease, or cure a disease. For example, the effective amount may be sufficient to achieve the effect of reducing the number of cancer cells, reducing the number of cells that are disrupted or chronically infected by the virus, or suppressing the growth and/or proliferation of the cells.

The effective amount may vary depending on a number of factors such as pharmacokinetic properties of a virus, an administration method, an age, a health condition and body weight of a patient, the characteristics and extent of a disease state, the number of treatments, and the most recent type of treatment, and may also vary depending on, for example, the virulence and titer of a virus. A person skilled in the art may adjust an appropriate amount based on the above factors. The virus may be initially administered in an appropriate dose as needed, depending on a patient's clinical response. The effective amount of virus may be empirically determined and may be determined by the maximum amount of virus that can be safely administered and the minimum amount of virus that cause a preferable result.

The concentration of an administered virus may vary depending on the virulence of a reovirus strain to be administered and the characteristics of target cells.

In the present invention, the reovirus may be included at 10² to 10¹⁵ PFU. In addition, the reovirus may be included at 10² to 10¹⁵ TCID₅₀, 10³ to 10¹⁴ TCID₅₀, 10³ to 10¹³ TCID₅₀, 10³ to 10¹² TCID₅₀, 10⁴ to 10¹² TCID₅₀, 10⁵ to 10¹¹ TCID₅₀, 10⁵ to 10¹⁰ TCID₅₀, 10⁶ to 10¹⁰ TCID₅₀, or 10⁷ to 10¹⁰ TCID₅₀.

The effective amount of virus may be administered repeatedly depending on the effect of the initial treatment regimen. Generally, the virus may be periodically administered while monitoring all responses. Those of ordinary skill in the art can easily grasp that a higher or lower dose of virus than that indicated above can be administered depending on the dosing schedule and a selected route.

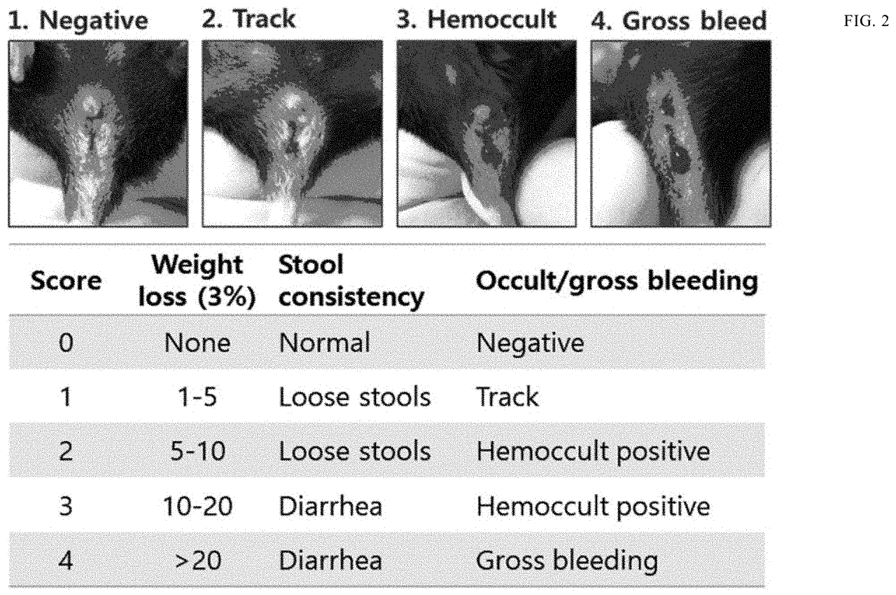

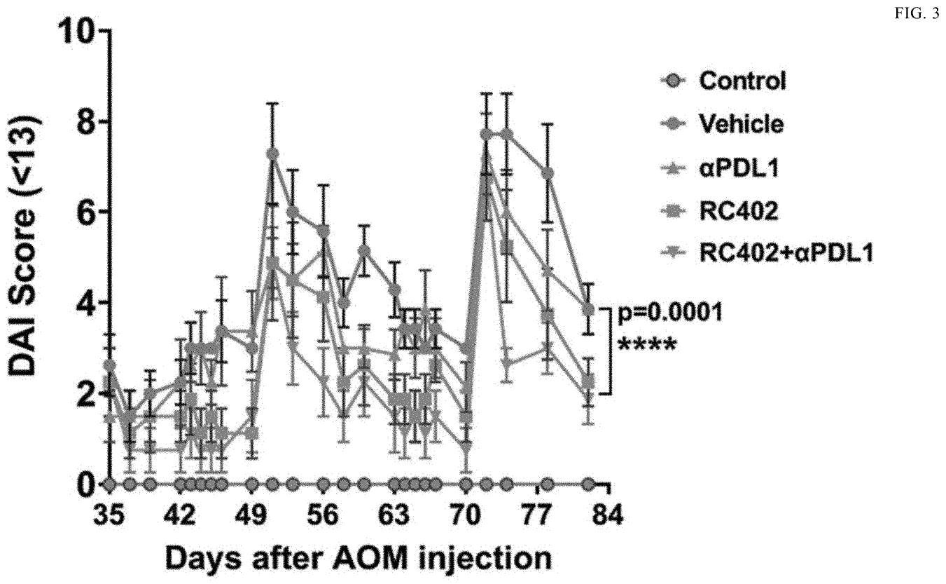

In one embodiment of the present invention, it was confirmed that the severity of colorectal cancer-related disease is significantly alleviated when a reovirus is administered alone or in combination with an anti PD-L1 antibody in a colorectal cancer animal model (FIGS. 2 and 3).

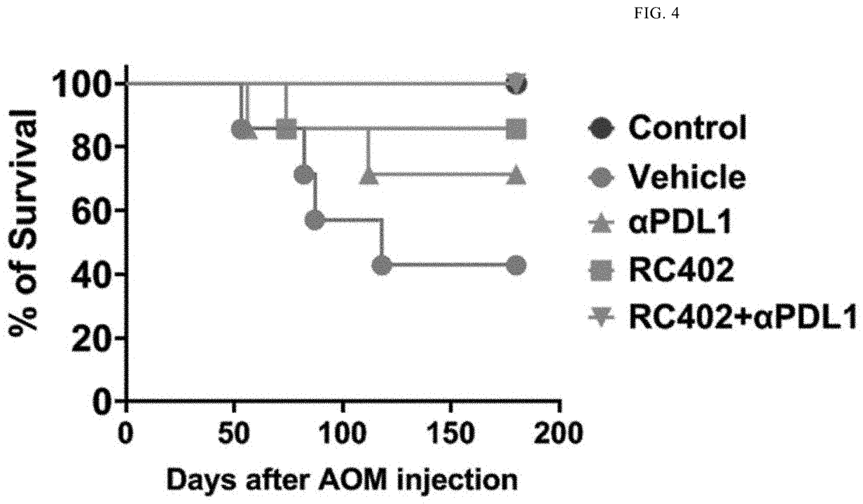

In another embodiment of the present invention, it was confirmed that the survival rate of a colorectal cancer animal model significantly increases when a reovirus is administered alone or in combination with an anti PD-L1 antibody in the colorectal cancer animal model (FIG. 4).

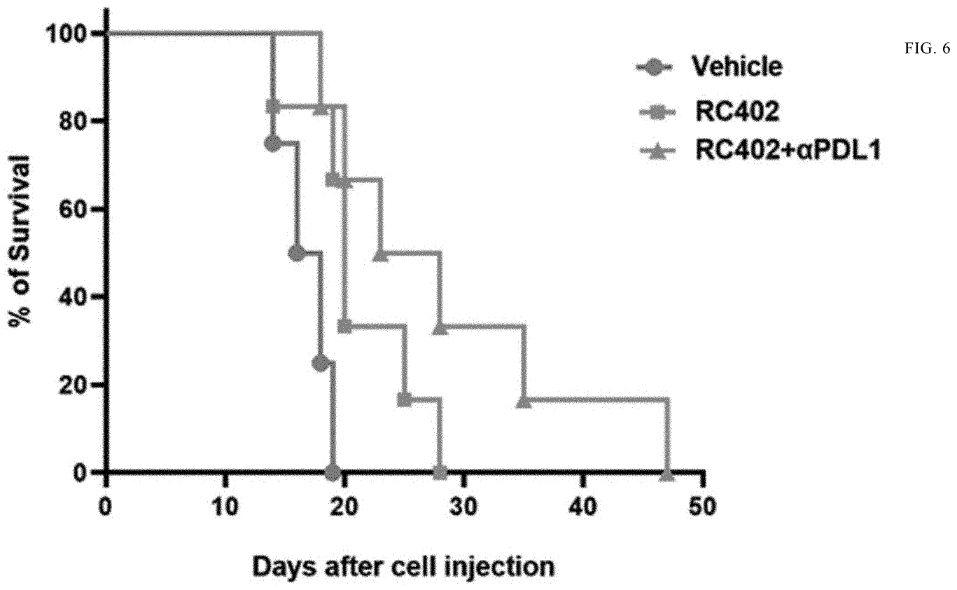

In still another embodiment of the present invention, it was confirmed that the survival rate of a colorectal cancer animal model significantly increases when reovirus is administered alone or in combination with an anti PD-L1 antibody in the colorectal cancer animal model (FIG. 6).

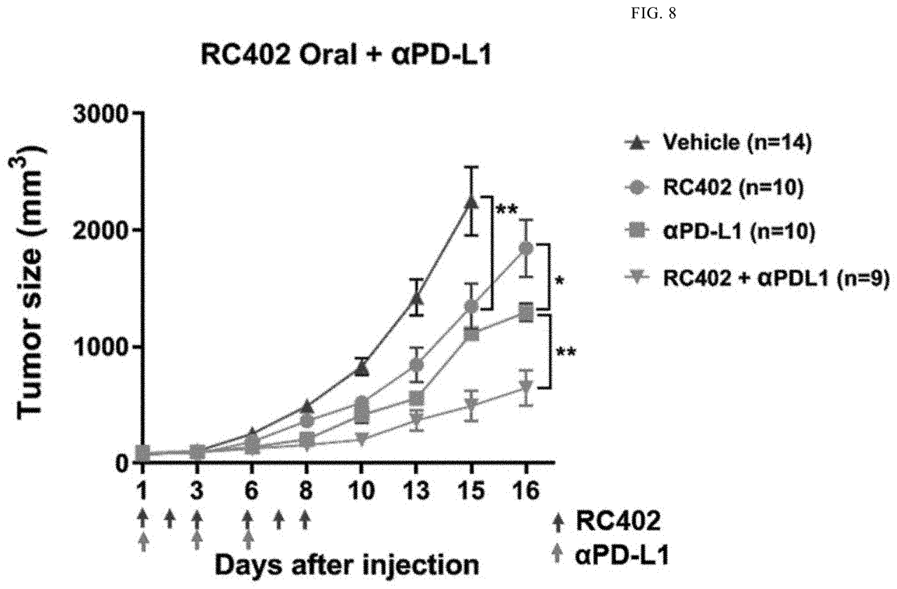

In yet another embodiment of the present invention, it was confirmed that the effect of reducing the volume of skin cancer significantly increases according to the co-administration of a reovirus and an anti PD-L1 antibody in a skin cancer animal model (FIG. 8).

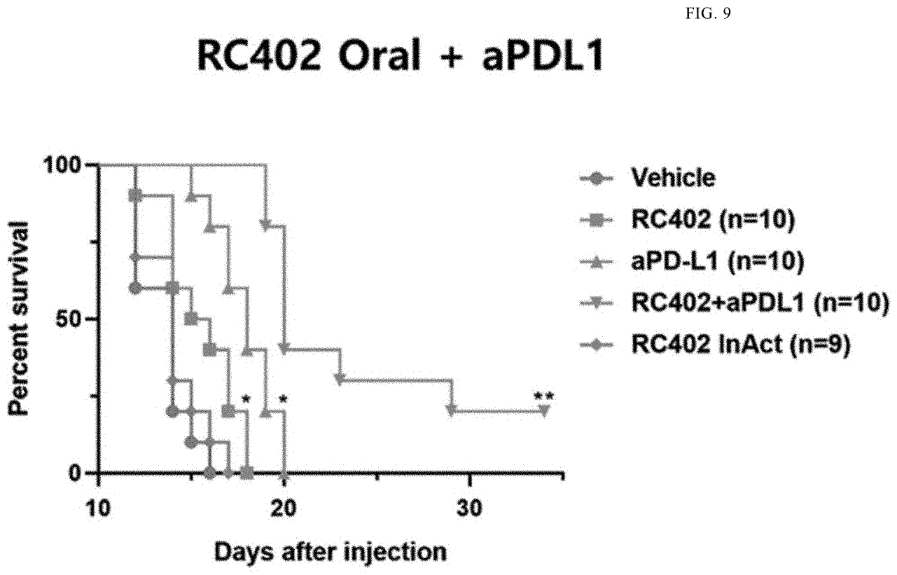

In yet another embodiment of the present invention, it was confirmed that the survival rate of a skin cancer animal model significantly increases according to the co-administration of a reovirus and an anti PD-L1 antibody in a skin cancer animal model (FIG. 9).

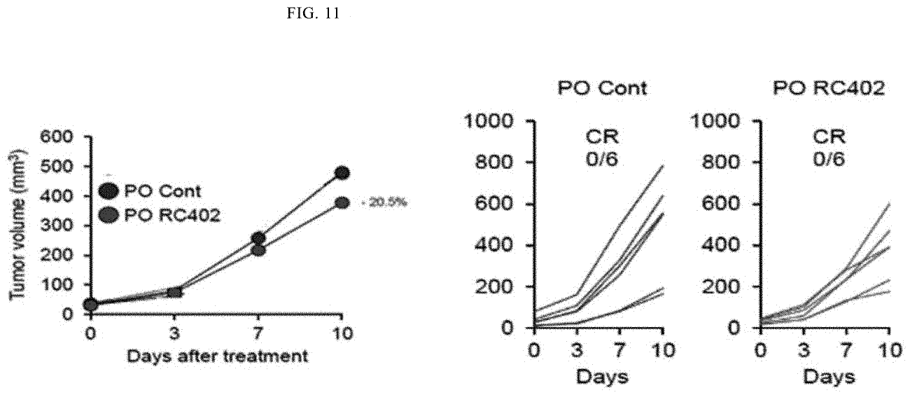

In yet another embodiment of the present invention, it was confirmed that the effect of reducing the volume of renal cancer and the effect of reducing the growth rate of renal cancer significantly increase according to the co-administration of a reovirus and an anti PD-L1 antibody in the renal cancer animal model (FIG. 11).

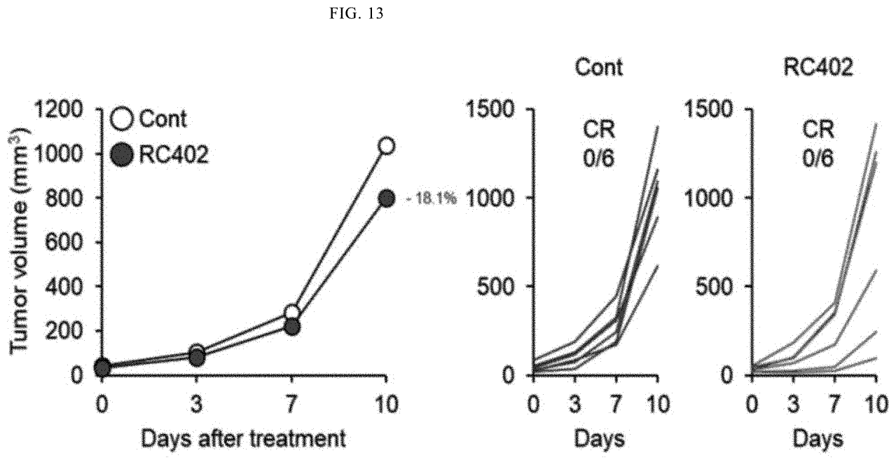

In yet another embodiment of the present invention, it was confirmed that the effect of reducing the volume of colorectal cancer and the effect of reducing a colorectal cancer growth rate significantly increase as a result of oral administration of a reovirus alone in the colorectal cancer animal model (FIG. 13).

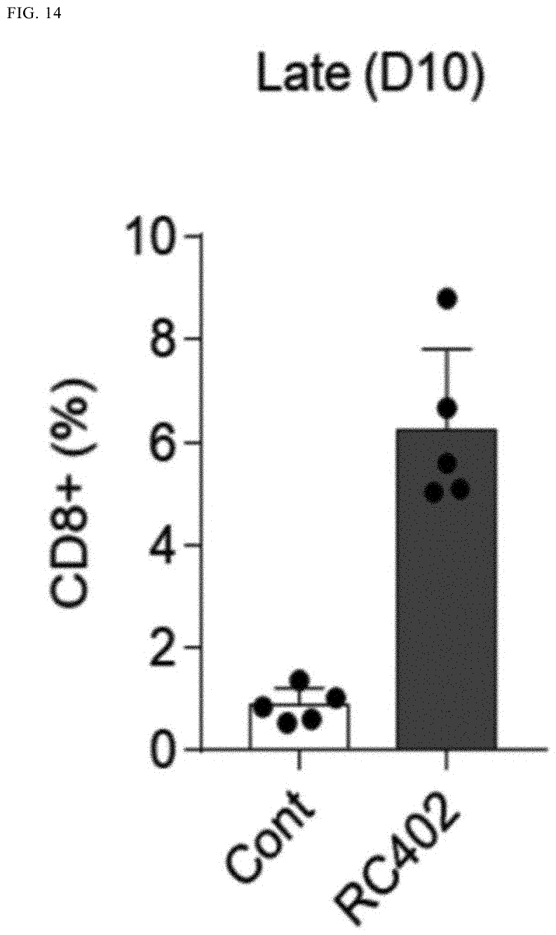

In yet another embodiment of the present invention, it was confirmed that the proportion of CD8+ T cells infiltrating into a tumor significantly increases as a result of oral administration of a reovirus alone in a colorectal cancer animal model (FIGS. 14 to 16).

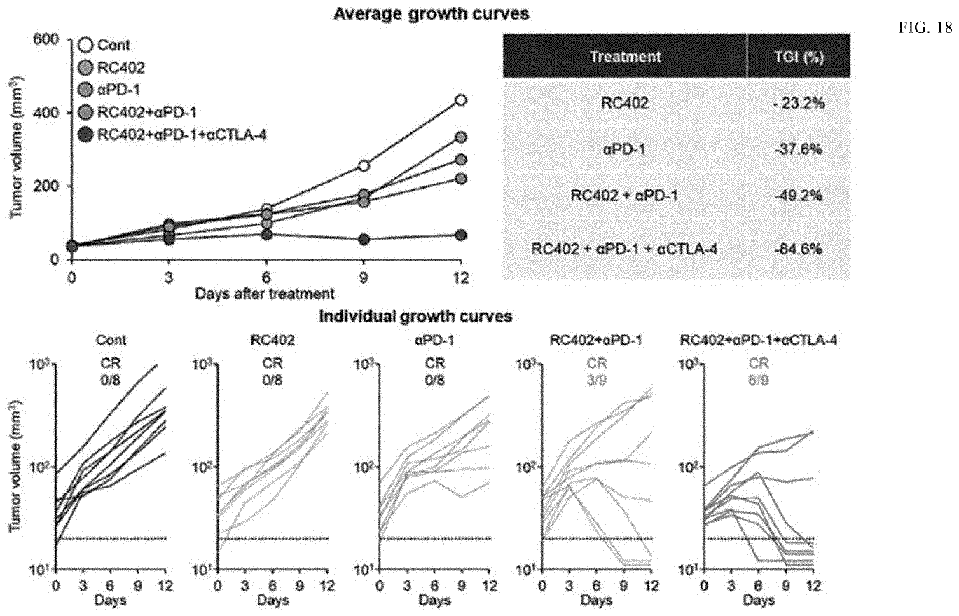

In yet another embodiment of the present invention, it was confirmed that the effect of reducing the volume of colorectal cancer and the effect of reducing a colorectal cancer growth rate significantly increase as a result of co-administration of a reovirus and an anti-PD-1 antibody and/or an anti-CTLA-4 antibody in the colorectal cancer animal model (FIG. 18).

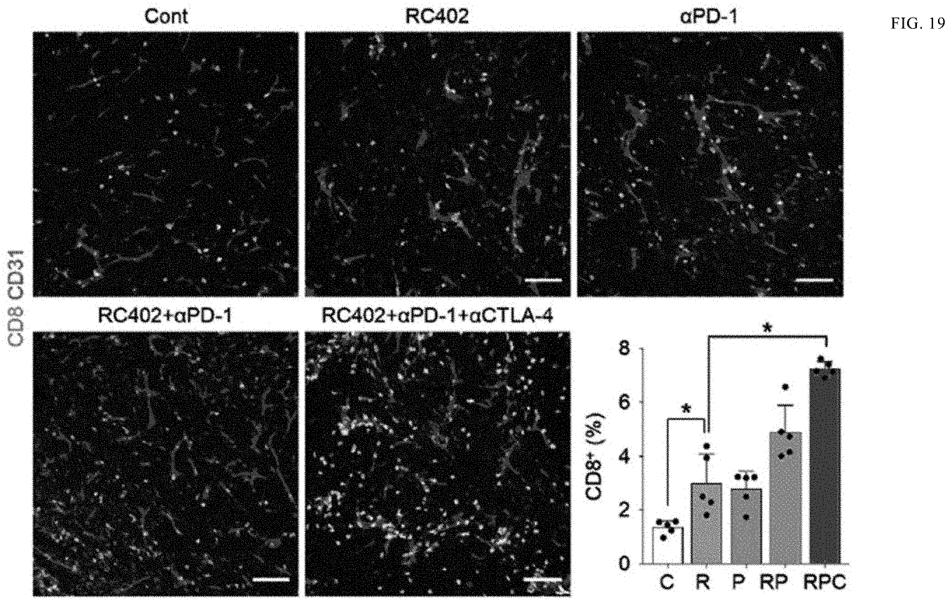

In yet another embodiment of the present invention, it was confirmed that the proportion of CD8+ T cells infiltrating into a tumor significantly increases as a result of co-administration of a reovirus and an anti-PD-1 antibody and/or an anti-CTLA-4 antibody in the colorectal cancer animal model (FIG. 19).

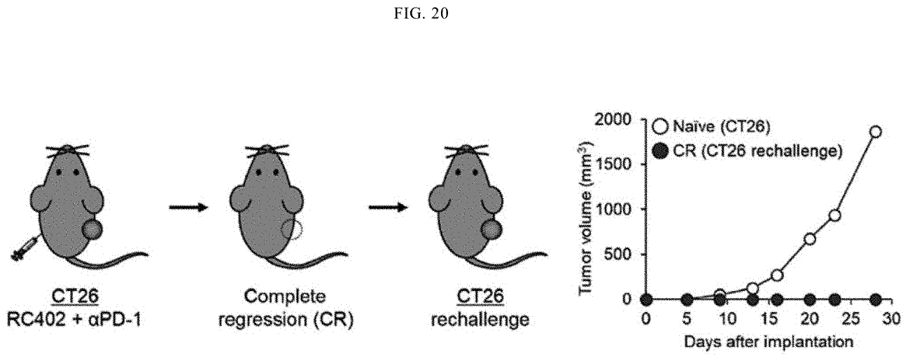

In yet another embodiment of the present invention, reovirus, it was confirmed that CT26 colorectal cancer does not recur as a result of CT26 rechallenge after the complete regression of colorectal cancer using a reovirus, an anti-PD-1 antibody and an anti-CTLA-4 antibody (FIG. 20).

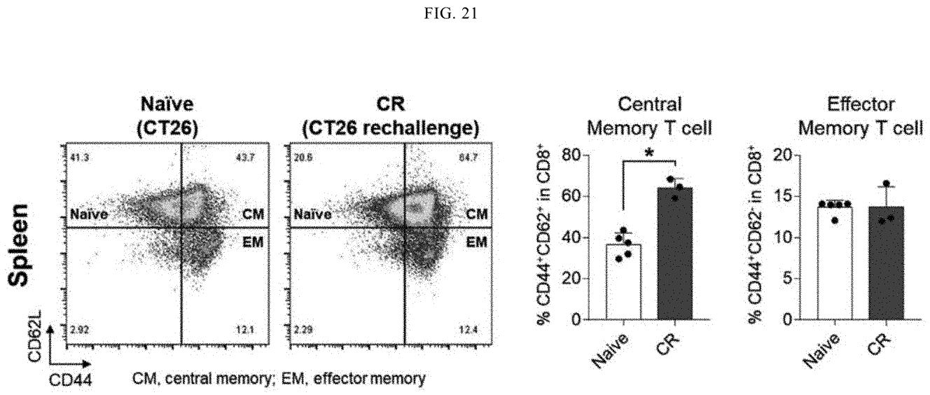

In yet another embodiment of the present invention, reovirus, it was confirmed that memory T cell rates in the spleen (CD44+CD62+CD8+) and the proportion of CD8+ T cells significantly increased as a result of CT26 rechallenge after complete regression of colorectal cancer using a reovirus, an anti-PD-1 antibody and an anti-CTLA-4 antibody (FIG. 21).

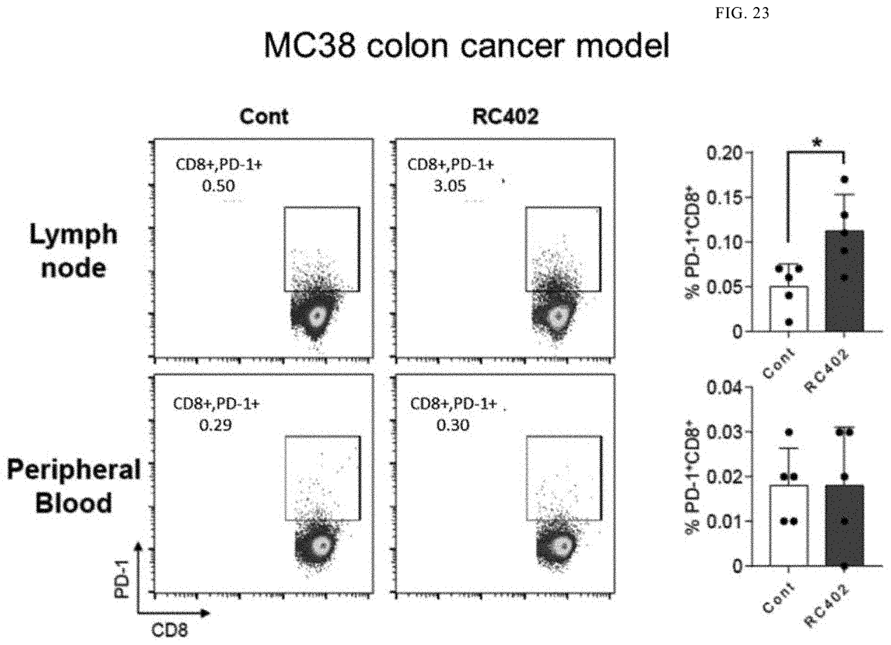

In yet another embodiment of the present invention, it was confirmed that PD-1 expression of CD8+ cells increased by reovirus administration in a mesenteric lymph node of a colorectal cancer model (FIG. 23).

According to the above results, it is expected that the pharmaceutical composition of the present invention can be used for preventing and treating cancer or recurrent cancer.

The pharmaceutical composition according to the present invention may be prepared with the reovirus; a reovirus-treated biological sample and an immune checkpoint inhibitor in the form of a single composition, or in the form of a separate composition. Preferably, the pharmaceutical composition according to the present invention may be prepared in the form of a separate composition. The method of preparing the same may use techniques commonly used in the art.

The total effective amount of the reovirus; and the reovirus-treated biological sample or the immune checkpoint inhibitor may be administered in a single dose, or by a fractionated treatment protocol in which multiple doses are administered over a long period of time. The pharmaceutical composition of the present invention may contain a different amount of components (a PD-L1 inhibitor, a PD-1 inhibitor, and/or a CTLA-4 inhibitor according to the present invention) depending on the severity of a disease and/or a purpose, and may be conventionally used at an effective dose of 0.01 μg to 10000 mg, and preferably 0.1 μg to 1000 mg for single administration and repeatedly administered several times a day. However, an effective dosage for a patient is determined in consideration of various parameters such as a formulation method, an administration route, the number of treatments as well as the patient's age, body weight, health condition and sex, the severity of a disease, a diet, and an excretion rate, so the suitable effective dosage of the composition of the present invention can be determined by those of ordinary skill in the art considering this point. The pharmaceutical composition according to the present invention is not particularly limited in its formulation, administration route and administration method as long as the effect of the present invention is exhibited.

The pharmaceutical composition according to the present invention may further include suitable carrier, excipient and diluent, which are conventionally used in preparation of a pharmaceutical composition. For example, the excipient may be one or more selected from the group consisting of a diluent, a binder, a disintegrant, a lubricant, an adsorbent, a humectant, a film-coating material, and a controlled-release additive.

The pharmaceutical composition according to the present invention may be formulated in the form of a powder, a granule, a sustained-release granule, an enteric granule, a solution and a liquid, an ophthalmic solution, an elixir, an emulsion, a suspension, a spirit, a troche, aromatic water, a lemonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a capsule, a perfusate, a plaster, a lotion, a paste, a spray, an inhalant, a patch, a sterile injection, or an external preparation such as an aerosol according to a conventional method, and the external preparation may be formulated in a cream, a gel, a patch, a spray, an ointment, a plaster, a lotion, a liniment, a paste or a cataplasma.

The carrier, excipient and diluent, which may be included in the pharmaceutical composition according to the present invention, may include lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil.

The pharmaceutical composition according to the present invention may be formulated with a diluent or an excipient such as a filler, a thickening agent, a binder, a wetting agent, a disintegrant, a surfactant, which are generally used in preparation.

As the additives for a tablet, powder, granule, capsule, pill and troche, excipients such as corn starch, potato starch, wheat starch, lactose, sucrose, glucose, fructose, di-mannitol, precipitated calcium carbonate, synthetic aluminum silicate, calcium monohydrogen phosphate, calcium sulfate, sodium chloride, sodium bicarbonate, purified lanolin, microcrystalline cellulose, dextrin, sodium alginate, methylcellulose, carboxymethylcellulose, sodium carboxymethylcellulose, kaolin, urea, colloidal silica gel, hydroxypropyl starch, hydroxypropyl methyl cellulose (HPMC), HPMC 1928, HPMC 2208, HPMC 2906, HPMC 2910, propylene glycol, casein, calcium lactate and Primojel; binders such as gelatin, gum arabic, ethanol, agar powder, cellulose acetate phthalate, carboxymethyl cellulose, carboxymethyl cellulose calcium, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch powder, hydroxypropylcellulose, hydroxypropylmethylcellulose, polyvinyl alcohol and polyvinylpyrrolidone; disintegrants such as hydroxypropylmethylcellulose, corn starch, agar powder, methylcellulose, bentonite, hydroxypropyl starch, sodium carboxymethylcellulose, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, gum arabic, amylopectin, pectin, sodium polyphosphate, ethyl cellulose, sucrose, magnesium aluminum silicate, a di-sorbitol solution and light anhydrous silicic acid; and lubricants such as calcium stearate, magnesium stearate, stearic acid, hydrogenated vegetable oil, talc, limestone kaolin, petrolatum, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, a higher fatty acid, a higher alcohol, silicone oil, paraffin oil, polyethylene glycol fatty acid ether, starch, sodium chloride, sodium acetate, sodium oleate, dileucine and light anhydrous silicic acid may be used.

As the additive for the liquid according to the present invention, water, diluted hydrochloric acid, diluted sulfuric acid, sodium citrate, sucrose monostearate, polyoxyethylene sorbitol fatty acid ester (Tween ester), polyoxyethylene monoalkyl ether, lanolin ether, lanolin ester, acetic acid, hydrochloric acid, aqueous ammonia, ammonium carbonate, potassium hydroxide, sodium hydroxide, prolamine, polyvinylpyrrolidone, ethyl cellulose, or sodium carboxymethylcellulose may be used.

As the syrup according to the present invention, a sucrose solution, other sugars or sweeteners may be used, and if needed, a fragrance, a coloring agent, a preservative, a stabilizer, a suspending agent, an emulsifier, or a thickening agent may be used.

As the emulsion according to the present invention, purified water may be used, and if needed, an emulsifier, a preservative, a stabilizer, or a fragrance may be used.

As the suspending agent according to the present invention, a suspending agent such as acacia, tragacanth, methylcellulose, carboxymethylcellulose, sodium carboxymethylcellulose, microcrystalline cellulose, sodium alginate, hydroxypropylmethylcellulose (HPMC), HPMC 1828, HPMC 2906, or HPMC 2910 may be used, and if needed, a surfactant, a preservative, a stabilizer, a coloring agent, or a fragrance may be used.

As the injection according to the present invention, a solvent such as injectable sterile water, 0.9% sodium chloride for injection, Ringer's solution, a dextrose for injection, dextrose+sodium chloride for injection, PEG, lactated Ringer's solution, ethanol, propylene glycol, non-volatile oil-sesame oil, cottonseed oil, peanut oil, soybean oil, corn oil, ethyl oleate, isopropyl myristic acid or benzene benzoate; a solubilizing agent such as sodium benzoate, sodium salicylate, sodium acetate, urea, urethane, monoethylacetamine, butazolidine, propylene glycol, Tween, nicotinamide, hexamine or dimethylacetamide; a buffer such as a weak acid and a salt thereof (acetic acid and sodium acetate), a weak base and a salt thereof (ammonia and ammonium acetate), an organic compound, a protein, albumin, peptone, or gums; an isotonic agent such as sodium chloride; a stabilizer such as sodium bisulfite (NaHSO₃), carbon dioxide gas, sodium metabisulfite (Na₂S₂O₃), sodium sulfite (Na₂SO₃), nitrogen gas (N₂) or ethylenediaminetetracetic acid; an antioxidant such as sodium bisulfide 0.1%, sodium formaldehyde sulfoxylate, thiourea, disodium ethylenediaminetetraacetate or acetone sodium bisulfite; a pain-relief agent such as benzyl alcohol, chlorobutanol, procaine hydrochloride, glucose or calcium gluconate; or a suspending agent such as sodium CMC, sodium alginate, Tween 80 or aluminum monostearate may be used.

As the suppository according to the present invention, a base such as cacao butter, lanolin, Witepsol, polyethylene glycol, glycerogelatin, methyl cellulose, carboxymethylcellulose, a mixture of stearate and oleate, Subanal, cottonseed oil, peanut oil, palm oil, cacao butter+cholesterol, lecithin, Lanette wax, glycerol monostearate, Tween or Span, Imhausen, monolene (propylene glycol monostearate), glycerin, Adeps solidus, Buytyrum Tego-G, Cebes Pharma 16, hexalide base 95, Cotomar, Hydrokote SP, S-70-XXA, S-70-XX75 (S-70-XX95), Hydrokote 25, Hydrokote 711, Idropostal, Massa estrarium, A, AS, B, C, D, E, I, T), Mass-MF, Masupol, Masupol-15, neosuppostal-N, paramount-B, supposiro (OSI, OSIX, A, B, C, D, H, L), suppository base IV types (AB, B, A, BC, BBG, E, BGF, C, D, 299), Suppostal (N, Es), Wecoby (W, R, S, M, Fs), or a Tegester triglyeride base (TG-95, MA, 57) may be used.

A solid formulation for oral administration may be a tablet, pill, powder, granule or capsule, and such a solid formulation may be prepared by mixing at least one excipient, for example, starch, calcium carbonate, sucrose, lactose or gelatin, with the active ingredient. Also, in addition to the simple excipient, lubricants such as magnesium stearate and talc may also be used.

As the liquid formulation for oral administration, a suspension, a liquid for internal use, an emulsion, or a syrup may be used, and a generally-used simple diluent such as water or liquid paraffin, as well as various types of excipients, for example, a wetting agent, a sweetener, a fragrance and a preservative may be included. A formulation for parenteral administration may be a sterilized aqueous solution, a non-aqueous solvent, a suspension, an emulsion, a lyophilizing agent or a suppository. As the non-aqueous solvent or suspension, propylene glycol, polyethylene glycol, a vegetable oil such as olive oil, or an injectable ester such as ethyl oleate may be used.

In the present invention, the pharmaceutical composition of the present invention may include albumin and a buffer, in addition to the active ingredient.

The pharmaceutical composition according to the present invention is administered at a pharmaceutically effective amount. The “pharmaceutically effective amount” used herein refers to an amount sufficient for treating a disease at a reasonable benefit/risk ratio applicable for medical treatment, and an effective dosage may be determined by parameters including a type of a patient's disease, severity, drug activity, sensitivity to a drug, administration time, an administration route and an excretion rate, the duration of treatment and drugs simultaneously used, and other parameters well known in the medical field.

The pharmaceutical composition according to the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without a side effect, and such a dose may be easily determined by one of ordinary skill in the art.

The pharmaceutical composition of the present invention may be administered into a subject via various routes. All methods for administration may be expected, and the pharmaceutical composition of the present invention may be administered by, for example, oral administration, subcutaneous injection, intraperitoneal administration, intravenous injection, intramuscular injection, paraspinal space (intrathecal) injection, sublingual administration, buccal mucosal administration, rectal insertion, vaginal insertion, ocular administration, ear administration, nasal administration, inhalation, spraying through a mouth or nose, skin administration, or transdermal administration.

The pharmaceutical composition of the present invention is determined by the type of a drug, which is an active ingredient as well as several related parameters such as a disease to be treated, an administration route, a patient's age, sex and body weight, and the severity of the disease.

The “subject” used herein refers to a subject in need of treatment of a disease, and more specifically, mammals such as a human or non-human primates, mice, rats, dogs, cats, horses, and cattle, but the present invention is not limited thereto.

The “administration” used herein refers to providing the composition of the present invention to a subject by any suitable method.

The “prevention” used herein refers to all actions of inhibiting or delaying the occurrence of a desired disease, the “treatment” used herein refers to all actions involved in alleviating or beneficially changing symptoms of a target disease and metabolic abnormality by the administration of the pharmaceutical composition according to the present invention, and the “improvement” refers to all actions of reducing parameters associated with a target disease, for example, the severity of symptoms by the administration of the composition according to the present invention.

All documents mentioned in the specification are incorporated herein by reference as described herein. When introducing elements of the present invention or preferred embodiment(s) thereof, the articles “a,” “an,” “the” and “said” are intended to mean one or more elements. The terms “comprising,” “including” and “having” are intended to be inclusive, and mean that there is an additional element other than the listed elements. Although the present invention has been described with reference to specific aspects or embodiments, it should not be construed as limiting the details of the aspects.

Hereinafter, to help in understanding the present invention, exemplary examples will be suggested. However, the following examples are merely provided to more easily understand the present invention, and not to limit the present invention.

### EXAMPLES

### Example 1

### Preparation of Azoxymethane (AOM)/Dextran Sulfate Sodium (DSS)-Induced Colorectal Cancer Animal Model

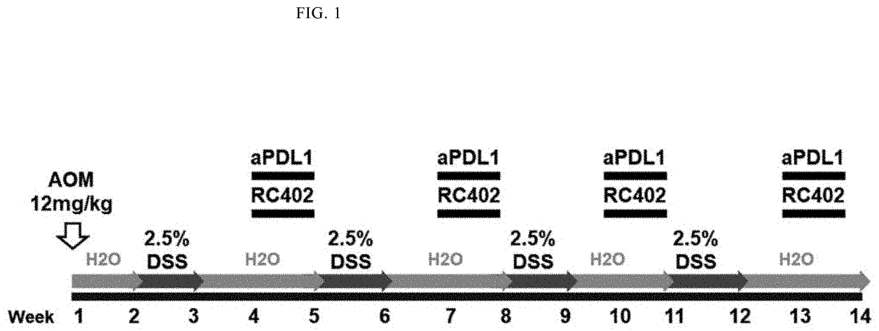

Animals used in an experiment were 7 to 8-week-old female BALB/C mice, purchased from Nara Biotech (Seoul, South Korea). The mice were acclimated for 7 days in an animal laboratory of ViroCure, Inc. and then subjected to the experiment, and during the acclimation period, water and feed were not limited. A standard environment was provided to the experimental animals, day and night were maintained at 12-hour intervals, and an indoor temperature was maintained at an appropriate level (23±2° C.). As shown in FIG. 1, colitis-associated colorectal cancer was induced by intraperitoneally injecting a carcinogen AOM (Merk, Cat #. 25843-45-2) into the mouse once at a concentration of 12 mg/kg, one week later, providing 2.5% (WN) DSS, which replaced common drinking water, for 1 week, and after a two-week rest period, repeating 4 sets of 1-week DSS treatment excluding AOM treatment, and 2-week rest periods.

### Example 2

### Oral Administration of Reovirus-Containing Composition and Systemic Administration of Immune Checkpoint Inhibitor (Anti-PD-L1 Antibody)

The mice used in the experiment were divided into a normal control, an AOM/DSS-induced colorectal cancer group, a reovirus oral administration group, an immune checkpoint inhibitor (anti-PD-L1 antibody) intraperitoneal administration group, and a reovirus/immune checkpoint inhibitor co-administration group, and subjected to the experiment. As a method for administering a reovirus into the colorectal cancer animal model, oral administration was used, and antibody administration was performed using direct intraperitoneal injection.

A reovirus (type 3, Dearing) was orally administered consecutively for 5 days as shown in FIG. 1 under conditions of 1×10⁸ TCID⁵⁰/100 μL PBS (Reovirus/0.1% (w/v) human serum albumin (HAS) in 1×PBS), and an anti-PD-L1 antibody (BioXcell, Cat #BE0101) was intraperitoneally administered three times every other day at 5 mg/kg. This administration was repeated a total of 4 times as shown in FIG. 1.

### Example 3

### Analysis of Therapeutic Effect of Reovirus-Containing Composition for Oral Administration Using AOM/DSS-Induced Colorectal Cancer Animal Model

From the 39^{th} day after the first AOM treatment, an experiment was performed while observing the presence or absence of colorectal cancer and the severity of symptoms through weight measurement, conditions of stool and the anus, a survival rate, and the formation and growth of cancer.

Statistical analysis was performed using GraphPad Prism 6. Among one-way ANOVA tests, a Dunnett's multiple comparison test was used to compare differences between a normal control, an AOM/DSS-induced colorectal cancer group, a reovirus oral administration group, an immune checkpoint inhibitor intraperitoneal administration group, and a reovirus/immune checkpoint inhibitor co-administration group. Differences with P values of less than 0.05 was considered statistically significant. Data was expressed as mean±SEM.

3-1. Visual Evaluation by Disease Activity Index (DAI) Measurement

To measure the severity of colorectal cancer-related diseases of colorectal cancer animal model mice, which were orally administered the reovirus-containing composition of Example 2 and intraperitoneally treated with an anti-PD-L1 antibody, disease activity was measured by checking a weight change, the firmness of stool, and the presence or absence of bloody stood visually observed in the stool or anus according to a grade of the disease activity index (DAI) based on that shown in FIG. 2.

As shown in FIG. 3, from day 4 after administration, soft stool and gross bloody stool began to appear in AOM/DSS-administered mice, and on day 7, diarrhea and bloody stools were observed in all mice. Meanwhile, in the co-administration group in which the reovirus was orally administered and the anti-PD-L1 antibody was intraperitoneally administered, it was confirmed that the degree of diarrhea and bloody stool was significantly improved.

This result shows that the co-administration of the reovirus-containing composition (oral administration) and the immune checkpoint inhibitor (e.g., anti-PD-L1 antibody) exhibits an excellent anticancer effect on colorectal cancer.

3-2. Evaluation by Measurement of Survival Rate

The colorectal cancer animal model leads to death through persistent inflammation in the colon and subsequent cancer formation.

As shown in FIG. 4, according to the progression of the disease in the colorectal cancer animal models, there were deaths in the AOM/DSS-induced colorectal cancer group and the single administration group (virus or antibody administration group), whereas no deaths occurred in the co-administration group.

These results show that the co-administration of the reovirus-containing composition (oral administration) and the immune checkpoint inhibitor (e.g., anti-PD-L1 antibody) can greatly improve a survival rate in colorectal cancer.

3-3. Evaluation by Measurement of Cancer Formation and Growth

According to the progression of the disease in the colorectal cancer animal models, in the single administration group (virus or antibody administration group), partial inhibition of cancer formation or an insignificant effect is expected. On the other hand, in the co-administration group, it is expected that cancer formation caused by inflammation will be greatly suppressed.

Therefore, the co-administration of the reovirus-containing composition (oral administration) and the immune checkpoint inhibitor (e.g., anti-PD-L1 antibody) is expected to greatly inhibit cancer formation and growth in colorectal cancer.

### Example 4

### Preparation of Colorectal Cancer Animal Model Through Surgical Implantation of Colorectal Cancer Cell Line (CT26) into Colon Wall

Animals used in an experiment were 7 to 8-week-old female BALB/C mice, purchased from Nara Biotech (Seoul, South Korea). The mice were acclimated for 7 days in an animal laboratory of ViroCure, Inc. and then subjected to the experiment, and during the acclimation period, water and feed were not limited. A standard environment was provided to the experimental animals, day and night were maintained at 12-hour intervals, and an indoor temperature was maintained at an appropriate level (23±2° C.). A colorectal cancer cell line (CT26 cells, 1×10⁶ cells/10 μL PBS) was injected into the colon wall and implanted into mice. A colorectal cancer animal model was established by confirming that cancer was formed 2 weeks after implantation.

### Example 5

### Oral Administration of Reovirus-Containing Composition and Systemic Administration of Immune Checkpoint Inhibitor (Anti-PD-L1 Antibody)

Animals used in an experiment were divided into a normal control, a CT26 cell implantation colorectal cancer group, a reovirus oral administration group, and a reovirus/immune checkpoint inhibitor co-administration group, and subjected to an experiment. A method of administering a reovirus into the colorectal cancer animal model was oral administration, and an antibody administration method was direct intraperitoneal injection.

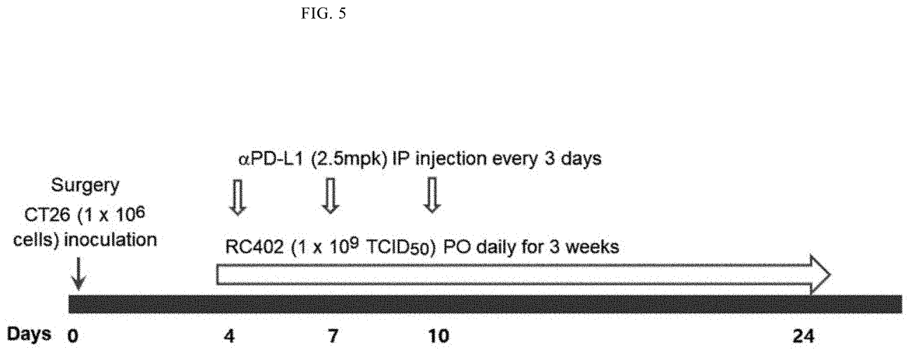

Four days after CT26 cell implantation, a reovirus (type 3, Dearing) was orally administered consecutively with 1×10⁹/100 μL of PBS (reovirus/0.1% (w/v) human serum albumin (HAS) in 1×PBS) for 3 weeks, and an anti-PD-L1 antibody (BioXcell, Cat #BE0101) was intraperitoneally administered three times every 3 days at 2.5 mg/kg (FIG. 5).

In the colorectal cancer animal model, as shown in FIG. 6, according to the progression of the disease, compared to the CT26 cell implantation colorectal cancer group, it was observed that the survival rate was improved in the treatment group (administration of virus alone or anti-PD-L1 antibody co-administration).

This result shows that the administration of the reovirus-containing composition alone (oral administration) or co-administration of the immune checkpoint inhibitor (e.g., anti-PD-L1 antibody) can greatly improve a survival rate in colorectal cancer.

### Example 6

### Preparation of Skin Cancer Animal Model by Implantation of Melanoma Cell Line (B16F10)

Animals used in an experiment were 5 to 8-week-old female BALB/C mice, purchased from Nara Biotech (Seoul, South Korea). The mice were acclimated for 7 days in an animal laboratory of ViroCure, Inc. and then subjected to the experiment, and during the acclimation period, water and feed were not limited. A standard environment was provided to the experimental animals, day and night were maintained at 12-hour intervals, and an indoor temperature was maintained at an appropriate level (23±2° C.). A melanoma cell line (B16F10 cells, 1×10⁵ cells/50 μL PBS) was subcutaneously injected into the flank of the mouse.

### Example 7

### Oral Administration of Reovirus-Containing Composition and Systemic Administration of Immune Checkpoint Inhibitor (Anti-PD-L1 Antibody)

An anticancer effect test was performed on a vehicle, a 0.1% (w/v) human serum albumin (HAS) in 1×PBS-administered control, a reovirus oral administration group, an immune checkpoint inhibitor (anti-PD-L1 antibody) intraperitoneal administration group, and a reovirus/immune checkpoint inhibitor co-administration group. A method of administering a reovirus into the colorectal cancer animal model was oral administration, and an antibody administration method was direct intraperitoneal injection.

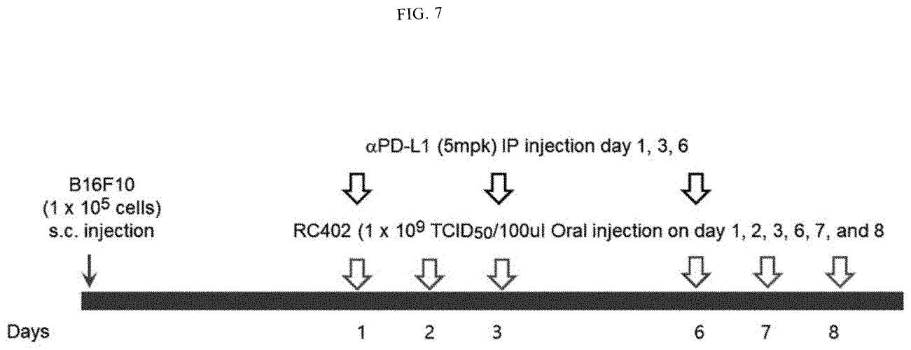

As shown in FIG. 7, one week after B16F10 cell implantation, a reovirus (type 3, Dearing) was orally administered with 1×10⁹ TCID₅₀/100 μL PBS (reovirus/0.1% (w/v) human serum albumin (HAS) in 1×PBS) six times on day 1, 2, 3, 6, 7, and 8, and an anti-PD-L1 antibody (Bioxcell, Cat #BE0101) was administered intraperitoneally 3 times every other day at 5 mg/kg.

### Example 8

### Analysis of Therapeutic Effect of Reovirus-Containing Composition for Oral Administration in Skin Cancer Animal Model

8-1. Evaluation by Measurement of Cancer Growth

As shown in FIG. 8, in the skin cancer animal model, it can be confirmed that the untreated control continued to grow after a tumor was generated in the mouse. As the disease progressed, in the single administration group (virus or antibody administration group), compared to the control, a cancer growth delay effect was observed. On the other hand, in the co-administration group, cancer growth was greatly inhibited.

Therefore, the co-administration of the reovirus-containing composition (oral administration) and the immune checkpoint inhibitor (e.g., anti-PD-L1 antibody) is expected to be effectively used for prevention or treatment of skin cancer.

8-2. Evaluation by Measurement of Survival Rate

As shown in FIG. 9, as the disease in the skin cancer animal model progressed, compared to the control and the single administration group (virus or antibody administration group), in the co-administration group, a survival rate was greatly improved.

Therefore, the co-administration of the reovirus-containing composition (oral administration) and the immune checkpoint inhibitor (e.g., anti-PD-L1 antibody) is expected to greatly improve a survival rate in skin cancer.

### Example 9

### Anticancer Effect of Single Administration in Renal Cancer Mouse Model

Animals used in an experiment were 5 to 8-week-old female BALB/C mice, purchased from Nara Biotech (Seoul, South Korea). A renal cancer cell line (RENCA cells, 2×10⁵ cells/50 μL PBS) was subcutaneously administered into the flank of the mouse.

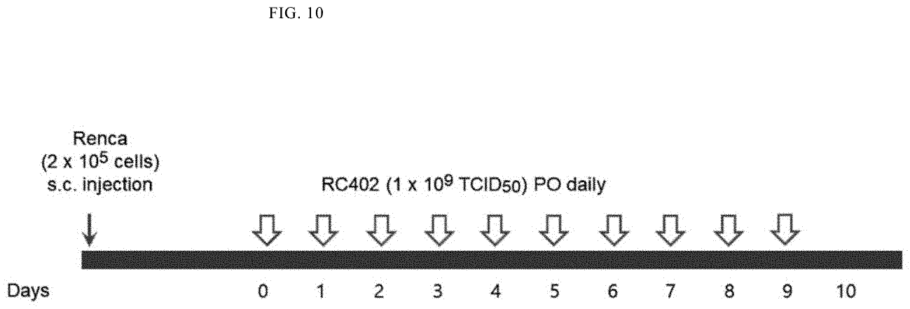

One week after RENCA cell implantation, a reovirus (type 3, Dearing) was orally administered under conditions of 1×10⁹ TCID₅₀/100 μL PBS (reovirus/0.1% (w/v) human serum albumin (HAS) in 1×PBS) once a day for 10 days (FIG. 10).

As shown in FIG. 11, it can be confirmed that the untreated control in the renal cancer animal models continued to grow after a tumor was generated in the mouse. In addition, as the disease progressed, a cancer growth delay effect was observed in the reovirus oral administration group compared to the control group.

### Example 10

### Induction of Increased Infiltration of Immune Cells into Tumor by Reovirus Oral Administration in Colorectal Cancer Cell Implanted Colorectal Cancer Animal Model

This example was to confirm in vivo whether an increase in infiltration of anticancer immune cells, CD8+ T cells, into a tumor is induced by oral administration of a reovirus.

10-1. Construction of Tumor Mouse Model

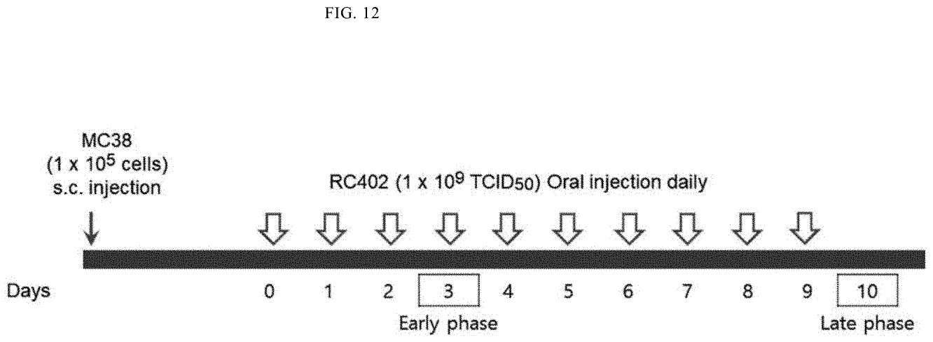

A MC38 cell line derived from C57BL6 colon adenocarcinoma cells was resuspended at a concentration of 1.0×10⁵ cells in 50 μL of PBS, and subcutaneously injected into the flank of a 6-week-old male C57BL6 mouse. One week after the MC38 cell implantation, a reovirus (type 3, Dearing) was orally administered under conditions of 1×10⁹ TCID₅₀/100 μL PBS (reovirus/0.1% (w/v) human serum albumin (HAS) in 1×PBS) once a day for 10 days (FIG. 12).

As shown in FIG. 13, in the colorectal cancer animal model, it can be confirmed that the untreated control continued to grow after a tumor was generated in the mouse. In addition, as the disease progressed, a cancer growth delay effect was observed in the reovirus oral administration group compared to the control.

10-2. Evaluation of Immune Cell Infiltration into Tumor

After three days and ten days of administration, a tumor was taken from the mouse, washed with PBS, cut into a size of 2 to 4 mm, and then treated with 1 ml of PBS containing 0.1 to 0.5% type I collagenase (Gibco, Cat No. P2031) at 37° C. for 30 minutes to 1 hour. Here, 10 ml of FACS buffer (0.1% BSA+0.01% sodium azide in PBS) was added and mixed and centrifuged at 300 to 400 g for 5 minutes. A supernatant was discarded and then the pellet was retrieved with 7 ml of FACS buffer, followed by filtering through a 70-μm cell strainer. The filtered single cells were mixed in 10 ml of FACS buffer, centrifuged at 300 to 400 g for 5 minutes to recover the precipitate. After discarding the supernatant, 1 to 2 mL of ACK lysis buffer (Lonza cat no. 10-548E) was added, left at room temperature for 1 minute, mixed in 10 ml of FACS buffer, and centrifuged at 300 to 400 g for 5 minutes. After resuspension with an appropriate amount of FACS buffer, the cells were stained with a CD8 monoclonal antibody (BD Biosciences, Cat no. 563234) to confirm the increasing level of CD8+ T cells, which was analyzed by flow cytometry using FACSDiVa software (BD Biosciences).

As shown in FIG. 14, after 10 days of administration, it was confirmed that the amount of the CD8+ T cells infiltrating into the tumors increased 5-fold or more in the reovirus oral administration group.

That is, through the increase in CD8+ T cells infiltrating into tumors, which serve as a representative biomarker of the tumor inhibitory effect of cancer immunotherapy, it is expected that a reovirus-containing composition for oral administration will act in cancer treatment by anticancer immune activation.

10-3. Evaluation of Immune Cell Infiltration into Tumor and Lymph Nodes

After three days and ten days of administration, tumors and lymph nodes were collected from mice, fixed with acetone, put in an OCT solution (Sakura Finetek, Cat No. 4583), and stored in a −80° C. cryogenic freezer to prepare a frozen tissue block. The frozen tissue block was cut into a thickness of 5 to 10 μm with a cryostat (Leica Biosystems, CM3050S) to manufacture a slide glass. A tissue section slide was washed with PBS using a Coplin Jar, covered with 200 μL Superblock (ThermoFisher Scientific, Cat no. 37515), and treated at room temperature for 10 minutes. Then, a fluorescence-labeled CD8 monoclonal antibody (Biolegend, Cat no. 100723) or CD31 monoclonal antibody (Biolegend, Cat no.102416), which was diluted in PBS, was stained at room temperature for 1 to 2 hours while blocking light, washed with PBS three times, coated with ProLong Diamond Antifade Mountant (ThermoFisher Scientific, Cat no. P36965), followed by covering with a coverslip. Afterward, the degree of staining was observed by a fluorescence microscope. Nuclear staining was conducted with a DAPI solution (BD Biosciences, Cat no. 564907) for 1 minute.

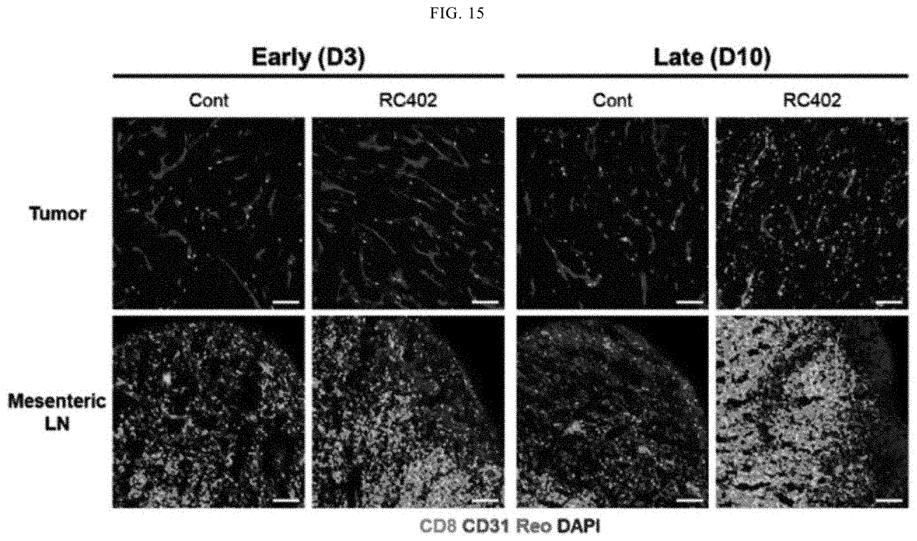

As shown in FIG. 15, it was confirmed that, after administration, the amount of CD8+ T cells infiltrating into the mesenteric lymph node showed a tendency to increase in the reovirus oral administration group, and particularly, the degree of increase on day 10 was remarkably high.

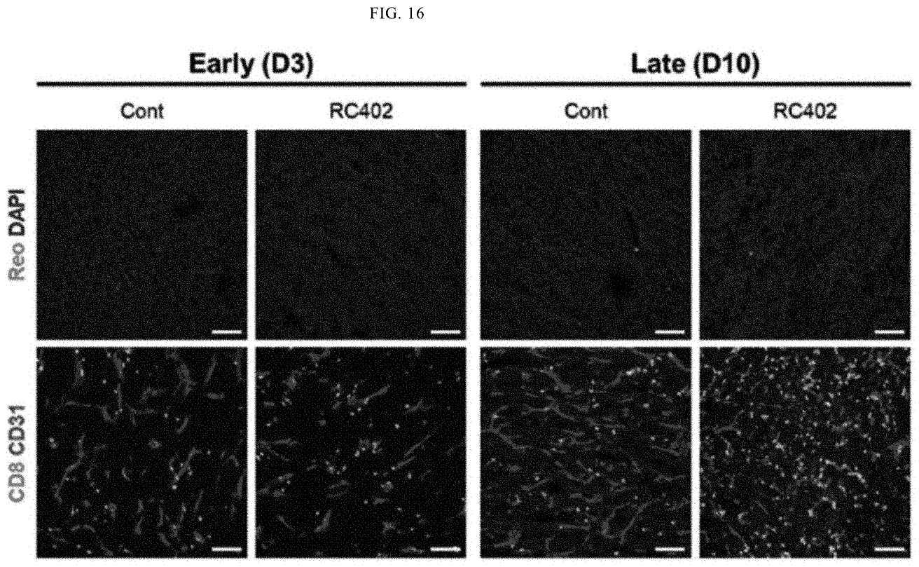

In addition, as shown in FIG. 16, on day 10 of administration, it was confirmed that the amount of CD8+ T cells infiltrating into the tumors was significantly increased in the reovirus oral administration group, which is the same as the flow cytometry analysis result.

Since the increase in CD8+ T cells infiltrating into the tumor is a representative biomarker of the tumor inhibitory effect of cancer immunotherapy, it is expected that the reovirus-containing composition for oral administration will act on cancer treatment through anticancer immune activation.

### Example 11

### Therapeutic Effect in Colorectal Cancer Animal Model

Animals used in an experiment were 5 to 8-week-old female BALB/C mice, purchased from Nara Biotech (Seoul, South Korea). The mice were acclimated for 7 days in an animal laboratory of ViroCure, Inc. and then subjected to the experiment, and during the acclimation period, water and feed were not limited. A standard environment was provided to the experimental animals, day and night were maintained at 12-hour intervals, and an indoor temperature was maintained at an appropriate level (23±2° C.). A colorectal cancer cell line (CT26 cells, 2×10⁵ cells/50 μL PBS) was subcutaneously injected into the flank of the mouse.

11-1. Analysis of Therapeutic Effect of Reovirus-Containing Composition for Oral Administration in Colorectal Cancer Animal Model

An anticancer effect test was performed on a vehicle, a 0.1% (w/v) human serum albumin (HAS) in 1×PBS-administered control, a reovirus oral administration group, an immune checkpoint inhibitor (anti-PD-L1 antibody) intraperitoneal administration group, and a reovirus/immune checkpoint inhibitor co-administration group. A method of administering a reovirus into the colorectal cancer animal model was oral administration, and an antibody administration method was direct intraperitoneal injection.

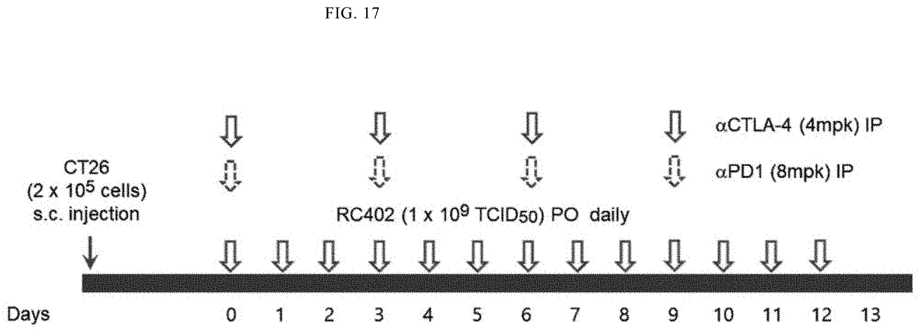

One week after CT26 cell implantation, a reovirus (type 3, Dearing) was orally administered under conditions of 1×10⁹ TCID₅₀/100 μL PBS (reovirus/0.1% (w/v) human serum albumin (HAS) in 1×PBS) once a day for 12 days. An anti-PD-1 antibody (Bioxcell, Cat #BE0033-2) was intraperitoneally administered at 8 mg/kg, and an anti-CTLA4 antibody (Bioxcell, Cat #BE0164) at 4 mg/kg four times every three days (FIG. 17).

11-2. Evaluation by Measurement of Cancer Growth

As shown in FIG. 18, it can be confirmed that the untreated control in the colorectal cancer animal models continued to increase in tumor volume after a tumor was generated in the mouse. However, as the disease progressed, in the single administration group (virus or antibody administration group), the cancer growth delay effect was observed compared to the control group.

On the other hand, in the co-administration group, and particularly, a treatment group in which two immune checkpoint inhibitors were co-administered (RC402+aPD-1+aCTLA-4), cancer growth was inhibited by 84.6% and a subject achieving complete regression (CR) was generated.

11-3. Evaluation of Immune Cell Infiltration into Tumor

After thirteen days of administration, a tumor was taken from the mouse, and the degree of infiltration of CD8+ T cells was comparatively analyzed using flow cytometry or immunohistochemistry.

As shown in FIG. 19, it was confirmed that the degree of tumor infiltration of CD8+ T cells in a virus-only or co-administration group compared to the control or antibody-only treated group increased in correlation with the degree of cancer growth inhibition, and particularly, it was confirmed that the degree of tumor infiltration of CD8+ T cells significantly increased in the treatment group in which two immune checkpoint inhibitors were simultaneously co-administered.

### Example 12

### Induction of Long-Term Anticancer Immunity for Preventing Cancer Recurrence after Treatment

12-1. Evaluation by Measurement of Cancer Growth

CT26 was re-implanted into a mouse in which a tumor was in complete regression through the co-administration of the reovirus-containing composition (oral administration) and the immune checkpoint inhibitor (anti-PD-1 antibody). As a control, a new BALB/C mouse was used.

As shown in FIG. 20, it was confirmed that, in the control group, the CT26 colorectal cancer cell line was normally grown, but when the CT26 colorectal cancer cells were re-implanted after complete regression, a tumor was not grown. This means that the anticancer immune effect lasts.

Therefore, the co-administration of the reovirus-containing composition (oral administration) and an immune checkpoint inhibitor (e.g., anti-PD-1 antibody, anti-CTLA4 antibody) is expected to clinically activate long-term anticancer immunity.

12-2. Analysis of Immune Cells in Spleen

10 ml of the spleen taken from the mouse in which complete regression had occurred was disrupted and then filtered through a 70-μm cell strainer. The resulting filtrate was retrieved by centrifugation at 300 to 400 g for 5 minutes. After discarding the supernatant, 1 to 2 mL of ACK lysis buffer (Lonza cat no. 10-548E) was added, left at room temperature for 1 minute, mixed in 10 mL of FACS buffer, and centrifuged at 300 to 400 g for 5 minutes. After resuspension with an appropriate amount of FACS buffer, the cells were stained with a CD8 monoclonal antibody (BD Biosciences, Cat no. 563234), CD44 antibody (Biosciences, Cat no. 560569), CD62 antibody (BD Biosciences, Cat no. 553150) to confirm the increasing level of CD8+ T cells, which was analyzed by flow cytometry using FACSDiVa software (BD Biosciences).

As shown in FIG. 21, it was confirmed that the proportion of memory T cells (CD44+Cd62+CD8+) increases in the CT26 colorectal cancer cell line-implanted mouse spleen. Therefore, it was determined that memory T cells are involved in long-term anticancer immunity.

That is, through the increase in CD8+ T cells, it is expected that the tumor-suppressive effect of cancer immunotherapy can activate long-term anticancer immunity such as prevention of recurrence.

### Example 13

### Immune Response by Reovirus Oral Administration

A MC38 cell line was resuspended at 1.0×10⁵ cells in 50 μL PBS, and subcutaneously injected into the flank of a 6-week-old male C57BL6 mouse. One week after the implantation of the MC38 cell line, a reovirus (type 3, Dearing) was orally administered once a day for 10 days under conditions of 1×10⁹ TCID₅₀/100 μL PBS (reovirus/0.1% (w/v) human serum albumin (HAS) in 1×PBS).

13-1. Increase in Tumor Antigen-Specific CD8+ T Cells

After ten days of administration, the mesenteric lymph node was taken from a mouse, and an MHC complex (MBL, Cat No. TS-M507) synthesized in the form of an MC38-specific antigen peptide KSPWFTTL-loaded tetramer was used to analyze the change in CD8+ T cells expressing T cell receptors (TCRs) specifically binding thereto through flow cytometry.

The mesenteric lymph node taken from the mouse was washed with PBS, gently disrupted, and then filtered through a 70-μm cell strainer. The filtered single cells were mixed in a 10 ml of FACS buffer, and retrieved by centrifugation at 300 to 400 g for 5 minutes. The cells were resuspended with an appropriate amount of FACS buffer at a concentration of 2×10⁷ cells/ml. Thereamong, 50 μL of the cell suspension was transferred to a test tube containing 10 μL of a Clear Back solution, and left at room temperature for 5 minutes. Here, 10 μL of T-Select MHC tetramer was added and then the resulting product was gently stirred, followed by freezing for 30 to 60 minutes or reacting under a light-shielded condition for 30 minutes. A monoclonal antibody (BD Biosciences, Cat no. 563234) against CD8 was added and reacted again under a light-shielded condition for 30 minutes. A supernatant was removed by centrifugation at 400 g for 5 minutes, the precipitate was resuspended in a phosphate buffer solution containing 0.5% paraformaldehyde or formalin. After leaving for 1 to 24 hours in a light-shielded refrigeration condition, the resulting product was analyzed by flow cytometry using FACSDiVa software (BD Biosciences). The frequencies of the MHC tetramer and the CD8+ T cells were expressed as a percentage of the total CD8+ T cells.

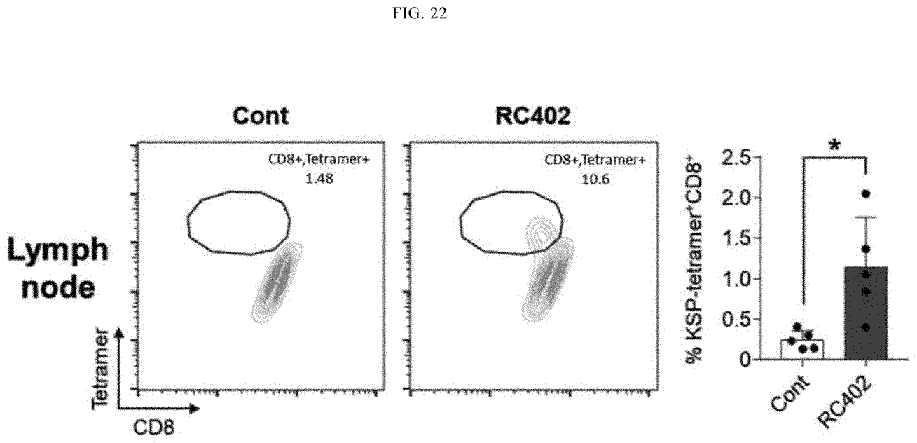

As shown in FIG. 22, it was confirmed that CD8+ T cells (KSP-tetramer+CD8+) with tumor antigen specificity were significantly increased in the administration group administered the reovirus-containing composition for oral administration.

13-2. Increasing PD-1 Expression in CD8+ T Cells in Lymph Node

The mesenteric lymph node was taken, washed with PBS, gently disrupted, and then filtered through a 70 μm cell strainer. The filtered single cells were mixed in 10 mL of FACS buffer, and then retrieved by centrifugation at 300 to 400 g for 5 minutes.

Blood was added to an EDTA (or heparin)-coated tube and mixed with PBS at 1:1, and the resulting mixture was transferred to a 15 mL tube containing Ficoll-Paque PLUS and centrifuged to retrieve mouse peripheral blood mononuclear cells (PBMCs). The PBMCs were centrifuged at 400 g to remove a supernatant, and 1 to 2 mL of ACK lysis buffer (Lonza cat no. 10-548E) was added and left at room temperature for 1 minute. Afterward, 10 mL of FACS buffer was added and mixed, and retrieved by centrifugation at 300 to 400 g for 5 minutes. Immune cells collected by isolation from a lymph node or blood were resuspended with an appropriate amount of FACS buffer, and stained with a CD8 monoclonal antibody (BD Biosciences, Cat no. 563234) and a monoclonal antibody (Biosciences, Cat no. 25-9985-80) against PD-1 to confirm the increase in CD8+ T cells expressing PD-1 using flow cytometry.

As shown in FIG. 23, it can be confirmed that the change in CD8+ T cells expressing PD-1 in PBMCs isolated from the blood was not observed, but the change in the mesenteric lymph node was evident, resulting in a 2.5-fold or more increase in CD8+ T cells.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not restrictive in any aspect.

### INDUSTRIAL APPLICABILITY

The present inventors not only confirmed the cancer treatment effect of reoviruses alone, but also confirmed significant increases in cancer treatment effects such as tumor volume reduction, inhibition of tumor growth rate, and an increase in survival rate for colorectal cancer, skin cancer, and renal cancer when reoviruses are orally administered and used in combination with an immune checkpoint inhibitor, and particularly, when a PD-1 antibody, a CTLA-4 antibody, and reoviruses are used in combination, the prevent inventors confirmed that not only cancer goes into complete regression, but also the recurrence of cancer can be inhibited. Therefore, the reovirus of the present invention will be effectively used as a therapeutic agent and therapeutic adjuvants for various types of cancer, so it has industrial availability.

1. A method of treating cancer or recurrent cancer, comprising:
administering a composition comprising a reovirus; or a reovirus-treated biological sample as an active ingredient into a subject.

2. The method of claim 1, wherein the cancer is one or more selected from the group consisting of cervical cancer, lung cancer, pancreatic cancer, non-small cell lung cancer, liver cancer, colon cancer, colorectal cancer, bone cancer, skin cancer, head and neck cancer, skin melanoma, intraocular melanoma, uterine cancer, ovarian cancer, rectal cancer, brain cancer, blood cancer, stomach cancer, perianal cancer, breast cancer, fallopian tube carcinoma, endometrial carcinoma, vaginal cancer, vulvar carcinoma, Hodgkin's disease, esophageal cancer, small intestine cancer, endocrine gland cancer, thyroid cancer, parathyroid cancer, adrenal cancer, soft tissue sarcoma, urethral cancer, penile cancer, prostate cancer, bladder cancer, kidney cancer, urinary tract cancer, renal cell carcinoma, renal pelvic carcinoma, CNS central nervous system (CNS) tumors, primary CNS lymphoma, spinal cord tumors, brainstem glioma, and pituitary adenoma.

3. The method of claim 1, further comprising an immune checkpoint inhibitor.

4. The method of claim 3, wherein the immune checkpoint inhibitor is one or more selected from the group consisting of a PD-L1 inhibitor, a PD-1 inhibitor, and a CTLA-4 inhibitor.

5. The method of claim 3, wherein the reovirus or the reovirus-treated biological sample; and the immune checkpoint inhibitor are simultaneously, separately or sequentially administered.

6. The method of claim 1, wherein the reovirus or the reovirus-treated biological sample is orally administered.

7. The method of claim 1, wherein the pharmaceutical composition increases infiltration of CD8+ T cells into a tumor.

8.-13. (canceled)
